(19) United States
(12) Patent Application Publication
(10) Pub. No.: US 2025/0019334 A1
(43) Pub. Date: Jan. 16, 2025

(54) HYDROFORMYLATION PROCESS FOR PREPARING LINEAR AND BRANCHED ALDEHYDES

(71) Applicant: NORTH CAROLINA STATE UNIVERSITY, Raleigh, NC (US)

(72) Inventors: Milad Abolhasani, Raleigh, NC (US); Malek Y.S. Ibrahim, Raleigh (NC)

(21) Appl. No.: 18/577,060

(22) PCT Filed: Jul. 7, 2022
(86) PCT No.: PCT/US2022/036380

§ 371 Date: Jan. 5, 2024

Related U.S. Application Data
(60) Provisional application No. 63/219,213, filed on Jul. 7, 2021.
(60) Provisional application No. 63/248,290, filed on Sep. 24, 2021.

(51) Int. Cl. C07C 45/50; B01J 23/46; B01J 31/22

(52) U.S. Cl. CPC C07C 45/50; B01J 23/464; B01J 31/2221; B01J 2231/321; B01J 2531/822

(57) ABSTRACT

Described are processes for preparing linear and branched aldehydes, which comprises reacting an olefin with hydrogen and carbon monoxide in the presence of a catalyst solution comprising rhodium, a hydroformylation solvent, and a fluorophosphite ligand having the general formula (I).

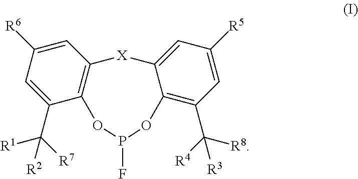

### CROSS-REFERENCE TO RELATED APPLICATION(S)

This application claims priority to U.S. Provisional Patent Application No. 63/219,213 filed on Jul. 7, 2021, and U.S. Provisional Patent Application No. 63/248,290 filed Sep. 24, 2021, the entire contents of which are incorporated herein by reference.

### TECHNICAL FIELD

The present disclosure generally relates to fluorophosphite ligands, catalyst solutions comprising the same, and hydroformylation processes for preparing linear and branched aldehydes employing the catalyst solutions.

### INTRODUCTION

Hydroformylation, also known as the oxo reaction, is one of the most widely practiced homogeneous catalyzed reactions in industry. Aldehydes produced by hydroformylation are routinely used in commodity chemicals, fragrances, and pharmaceuticals. The hydroformylation reaction is used extensively in commercial processes for the preparation of aldehydes by the reaction of an olefin with hydrogen (H₂) and carbon monoxide (CO).

### SUMMARY

In one aspect, the present disclosure provides fluorophosphite ligands of formula (I):

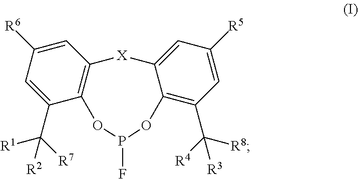

wherein:
X is —CH(R)—;
R is independently C₁₋₆alkyl, a 3- to 7-membered carbocycle, or a phenyl, where the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl;
R¹, R², R³, R⁴ are each independently C₁₋₆alkyl;
R⁵ and R⁶ are each

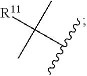

R¹¹ is a C₁₋₆alkyl, a 3- to 6-membered carbocycle, or a phenyl, wherein the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl; and
R⁷ and R⁸ are each independently C₁₋₆alkyl, a 6- to 12-membered aryl, or a 3- to 6-membered carbocycle, wherein the 6- to 12-membered aryl and 3- to 6-membered carbocycle are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl.

In other aspects, the present disclosure provides processes for preparing linear and branched aldehydes, the processes comprising contacting an olefin with hydrogen (H₂) and carbon monoxide (CO) in the presence of a catalyst solution.

The contacting of the olefin with the H₂ and the CO in the presence of the catalyst solution at a temperature ranging from 95° C. to 130° C., a carbon monoxide partial pressure (P_{CO}) ranging from 110 pounds per square inch absolute (psia) to 350 psia, and a hydrogen partial pressure (_{H2}) ranging from 20 psia to 150 psia, may produce a ratio of linear aldehydes to branched aldehydes (l/b) of less than 1.0.

The contacting of the olefin with the H₂ and the CO in the presence of the catalyst solution at a temperature ranging from 60° C. to 100° C., a P_{CO} ranging from 5 psia to 150 psia, and a P_{H2} ranging from 100 psia to 350 psia, may produce l/b of greater than 1.0.

### BRIEF DESCRIPTION OF THE DRAWINGS

FIG. 1 is a schematic illustration of the flow chemistry platform utilized for mechanistic studies of olefin hydroformylation reactions.

FIG. 2A-2C are graphs illustrating the total aldehyde yield, internal isomer yield, and aldehyde regioselectivity, i.e., l/b=l/(b1+b2+b3), at different reaction (i.e., residence) times, t_{R}, in the flow reactor and at a 2 h reaction time in the batch reactor. Reaction conditions: [1-octene]=0.5 M, [Rh]=0.05 mol %, L:Rh=10:1, Pₜₒₜₐₗ=200 pounds per square inch absolute (psia), H₂/CO=1. (PA) indicates catalyst pre-activation.

FIG. 2A is a graph showing the total aldehyde yield in the continuous flow (0<t_{R}<50 min) and the batch (2 h) reactors with L.

FIG. 2B is a graph illustrating the internal octene isomer yield in the continuous flow (0<t_{R}<50 min) and the batch (2 h) reactors with L.

FIG. 2C is a graph showing the regioselectivity of 1-octene hydroformylation in the continuous flow (0<t_{R}<50 min) and the batch (2 h) reactors with L.

FIG. 3A-3C are graphs indicating the effects of reaction parameters CO pressure (P_{CO}), ligand concentration ([L]), and temperature (T) on the hydroformylation of 1-octene at fractional conversion. For all plots [1-octene]=0.5 M, Pₜₒₜₐₗ=350 psia, t_{R}=20 min, preactivated Rh+L catalyst: [Rh]=1 mmol. %.

FIG. 3A is a graph showing the turnover frequency (TOF) and l/b of 1-octene hydroformylation at variable P_{CO}. Reaction conditions: T=75° C., [L]=10 mM.

FIG. 3B is a graph illustrating the TOF and l/b of 1-octene hydroformylation at variable ligand concentration. Reaction conditions: T=75° C., P_{CO}=7 psia.

FIG. 3C is a graph showing the TOF and l/b of 1-octene hydroformylation at variable T. Reaction conditions: P_{CO}=7 psia, [L]=10 mM.

FIG. 4A-4C are graphs plotting the internal isomer yield, aldehyde yield, and TOF under variable CO pressure (P_{CO}) when 1-octene is reacted with Rh/L at 110° C. in the absence of H₂.

FIG. 4A is a graph showing the effect of P_{CO} on the isomerization of 1-octene with Rh/L catalyst at t_{R}=20 min in flow and 1 h in batch reactions. Conditions: T=110° C., [Rh]=0.05 mol %, L:Rh=10:1. [1-octene]=0.5 M.

FIG. 4B is a graph showing the effect of the P_{H2} at P_{CO}=85 psia and 110° C. on the hydroformylation of 1-octene with Rh/L catalyst in flow. Reaction conditions: T=110° C., [Rh]=0.05 mol. %, L:Rh=5:1, t_{R}=11 min, [1-octene]=0.5 M. The catalyst was pre-activated for these experiments.

FIG. 4C is a graph illustrating the effect of the P_{CO} on the hydroformylation of 0.5 M 2-octene (cis+trans). Reaction conditions: Pₜₒₜₐₗ=200 psia, [Rh]=0.02 mol. %, L:Rh=7:1, t_{R}=6.8 min, T=75° C. The catalyst was pre-activated for these experiments.

FIG. 5 is a graph showing the continuous on-demand switching from predominantly linear (area of graph to the left of the dotted line) to branched aldehyde (area of graph to the right of the dotted line) in the flow reactor (TOS denotes time on stream). Reaction conditions: Pₜₒₜₐₗ=350 psia, t_{R}=15 min, [1-octene]=0.05 M in toluene, and [L]=10 mM. G:L denotes the gas:liquid ratio.

FIG. 6 is a bar graph illustrating the regioselectivity ranges for Aldehyde regioselectivity flexibility ranges for 1-octene (top panel) and propylene (bottom panel) hydroformylation with a Rh catalyst preactivated with different ligands from Table 3. The numbers on top and bottom of bars in the top (1-octene) and bottom (propylene) panels correspond to the total aldehyde yields of the specific ligand under the linear-selective (l/b>1) and branched-selective (l/b<1) conditions. Linear-selective conditions (l/b>1): P_{CO}=7 psia, T=75° C., [Rh]=0.05 mol. %, Ligand:Rh=40:1 for L, L′-2, L′-3, and L′-4, and Ligand:Rh=1.1:1 for L′-5 and L′-6, t_{R}=60 min. Branched-selective conditions (l/b<1): P_{CO}=150 psia, T=110° C., [Rh]=0.05 mol. %, Ligand:Rh=2:1 for L, L′-2, L′-3, and L′-4, and Ligand:Rh=1.1:1 for L′-5 and L′-6, t_{R}=120 min. Pₜₒₜₐₗ=300 psia for both conditions. The 2-step hydroformylation reaction with L is at Scheme 2, entry (3) conditions.

FIG. 7 is a graph showing plots of the TOF and l/b for 1-octene hydroformylation without any ligand under variable P_{CO}. Reaction conditions: T=75° C., [1-octene] in toluene=0.5 M, Pₜₒₜₐₗ=200 psia, t_{R}=6.8 min, pre-activated Rh(CO)₂acac catalyst, [Rh]=0.05 mol %.

FIG. 8 is a graph showing the TOF and l/b of 1-octene hydroformylation at variable [L] under P_{CO}=135 psia. Reaction conditions: T=80° C., [1-octene] in toluene=0.5 M, Pₜₒₜₐₗ=200 psia, t_{R}=4.5 min, pre-activated Rh/L catalyst: [Rh]=0.01 mol. %.

FIGS. 9A-9B are graphs illustrating the dependence of the aldehyde yield on the Rh loading in the hydroformylation of 1-octene in toluene with Rh/L catalyst in the flow reactor at P_{CO}=7 psia and Pₜₒₜₐₗ=350 psia. Reaction conditions: T=75° C., [L]=10 mM, t_{R}=21.8 min, pre-activated catalyst.

FIG. 9A shows the uncorrected TOF graph.

FIG. 9B shows the corrected TOF graph.

FIG. 10 is a graph showing plots of the dependence of the aldehyde yield on the [1-octene] concentration in the hydroformylation of 1-octene in toluene with Rh/L catalyst in the flow reactor. Reaction conditions: T=110° C., P_{CO}=100 psia, [R]=0.05 mol %, L:Rh=10:1, t_{R}=20 min.

FIG. 11 is a graph showing the ln(TOF) vs. 1/T for 1-octene hydroformylation at P_{CO}=107 psia and 65 psia. Reaction conditions: [1-octene] in toluene=0.5 M, Pₜₒₜₐₗ=200 psia, t_{R}=6.8 min, pre-activated Rh/L catalyst: [Rh]=0.01 mol. %.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### I. Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,”, “may,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” is used to indicate that exact values are not necessarily attainable. Therefore, the term “about” is used to indicate this uncertainty limit. The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5-1.4. The modifier “about” should also be considered as disclosing the range defined by the absolute values of the two endpoints. For example, the expression “from about 2 to about 4” also discloses the range “from 2 to 4.”

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

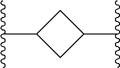 [SMILES: *C1CC(*)C1]

and

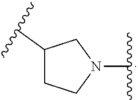 [SMILES: *C1CCN(*)C1]

Cycloalkylene and heterocyclylene include a geminal divalent groups such as 1,1-C₃₋₆cycloalkylene

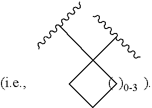 [SMILES: *C1(*)CCC1]

A further example is 1,1-cyclopropylene

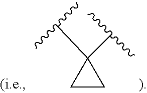 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven-and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13, 7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “hydroformylation,” as used herein is contemplated to include, but is not limited to, all hydroformylation processes that involve converting one or more substituted or unsubstituted olefinic compounds or a reaction mixture comprising one or more substituted or unsubstituted olefinic compounds to one or more substituted or unsubstituted aldehydes or a reaction mixture comprising one or more substituted or unsubstituted aldehydes. The aldehydes may be asymmetric or non-asymmetric.

The term “fractional conversion,” as used herein means the number of moles of a compound that reacted divided by the amount of the moles that were fed.

The term “free ligand,” as used herein means ligand that is not complexed with, tied to or bound to, the metal, e.g., metal atom, of the complex catalyst.

The term “Syngas,” (from synthesis gas) as used herein is the name given to a gas mixture that contains varying amounts of carbon monoxide (CO) and hydrogen (H₂). Production methods are well known and include, for example: (1) steam reforming and partial oxidation of natural gas or liquid hydrocarbons and (2) the gasification of coal and/or biomass. Hydrogen and CO typically are the main components of syngas, but syngas may contain carbon dioxide and inert gases such as nitrogen (N) and argon (Ar). The molar ratio of H₂ to CO varies greatly but generally ranges from 1:100 to 100:1 and may be between 1:10 and 10:1. Syngas is commercially available and is often used as a fuel source or as an intermediate for the production of other chemicals. The H₂:CO molar ratio for chemical production may be between 3:1 and 1:3 and usually is targeted to be between about 1:2 and 2:1 for most hydroformylation applications.

The term “catalyst solution” as used herein, may include, but is not limited to, a mixture comprising: (a) a metal-organophosphorous ligand complex catalyst, (b) free metal, (c) free organophosphorous ligand, and (d) a solvent for said metal-organophosphorous ligand complex catalyst, said free metal, and said free organophosphorous ligand.

The term “complex” as used herein and in the claims means a coordination compound formed by the union of one or more electronically rich molecules or atoms capable of independent existence with one or more electronically poor molecules or atoms, each of which is also capable of independent existence. The phosphite ligands employed herein, which possesses one phosphorus donor atom, have one available or unshared pair of electrons which may form a coordinate covalent bond independently or with another phosphite ligand in concert (e.g., via chelation) with rhodium (Rh). Carbon monoxide (which is also properly classified as a ligand) is also present and complexed with Rh.

### II. Linear vs. Branched (l/b) Selectivity

Generally, in hydroformylation processes, a transition metal-organophosphorus ligand complex catalyst produces an isomeric mixture comprising a linear (l, normal, or n-) aldehyde and one or more branched (b, iso-, or i-) aldehydes. A ratio of the linear aldehyde to the sum of the branched aldehydes, calculated by molar or by weight, is often described as l/b selectivity or l/b ratio. Since all isomeric aldehydes produced from a given olefinically-unsaturated compound have an identical molecular weight, the molar l/b ratio is identical to the weight l/b ratio. For the purposes of this disclosure, an l/b selectivity of a catalyst refers to the l/b ratio obtained from hydroformylation of an olefin unless otherwise stated.

In industry settings it is a labor-intensive process to shift from linear-selective reactions to branched-selective reactions. Typically, in industry, the aldehyde products are separated from the catalyst solution via distillation or extraction and said catalyst solution is recycled. However, when transitioning from branch-selective processes to linear-selective processes, the rhodium catalyst system may need to be altered, because a different organophosphorus ligand may be required to generate the opposite selectivity. This ligand-Rh disengagement is a costly process that poses a need for modular phosphine ligands that enable formation of both linear and branched aldehydes depending on simple and adjustable reaction parameters.

### III. Exemplary Compositions and Methods

The present disclosure provides hydroformylation processes that enable selective formation of linear and branched aldehyde products.

Broadly, exemplary processes comprise a fluorophosphite ligand, a catalyst solution comprising the same, and a regioselective hydroformylation process employing the catalyst solution. Aldehydes produced by hydroformylation processes may be referred to as “oxo aldehydes,” which have a wide range of utility, for example, as intermediates for hydrogenation to aliphatic alcohols, for amination to aliphatic amines, for oxidation to aliphatic acids, and for aldol condensation to plasticizers. Various aspects of exemplary processes, including descriptions of a fluorophosphite ligand, a catalyst solution comprising the same, and a regioselective hydroformylation process employing the catalyst solution, are discussed in the following sections.

The present disclosure provides processes for preparing linear and branched aldehydes, comprising contacting an olefin with hydrogen and carbon monoxide in the presence of a catalyst solution, under hydroformylation conditions, as herein described.

### A. Catalyst Solution

The disclosure further provides a highly active catalyst solution for use in regioselective hydroformylation reactions. A hydroformylation process comprises contacting under reaction conditions an olefin with carbon monoxide and hydrogen in the presence of a catalyst to produce one or more aldehydes.

Hydroformylation processes typically employ transition metal-organophosphorus ligand complex catalysts. The present disclosure employs transition metal-fluorophosphite ligand complex catalysts.

### 1. Transition Metal

Transition metals may include, but are not limited to, iron, ruthenium, osmium, cobalt, rhodium, iridium, nickel, palladium and platinum. Rhodium may be a preferred transition metal for some reactions.

Rhodium compounds that may be used as a source of rhodium for the active catalyst may include rhodium (II) or rhodium (III) salts of carboxylic acids, examples of which include di-rhodium tetraacetate dihydrate, rhodium (II) acetate, rhodium (II) isobutyrate, rhodium (II)₂-ethylhexanoate, rhodium (II) benzoate, and rhodium (II) octanoate. Also, rhodium carbonyl species such as Rh₄(CO)₁₂, Rh₆(CO)₁₆, and rhodium (I) acetylacetonate dicarbonyl may be suitable rhodium feeds. Other rhodium sources may include rhodium salts of strong mineral acids such as chlorides, bromides, nitrates, sulfates, phosphates, and the like.

### 2. Fluorophosphite Ligands of Formula (I)

In some aspects, the present disclosure provides compounds of formula (I), wherein X, R, R¹, R², R³, R⁴, R⁵, R⁶, R⁷, and R⁸ are as defined herein.

Unsubstituted or substituted rings (i.e., optionally substituted) such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

Fluorophosphite ligands that may be useful in the present disclosure are set forth in the following numbered embodiments. The first embodiment is denoted E1, the second embodiment is denoted E2 and so forth.

E1. A fluorophosphite ligand of formula (I):

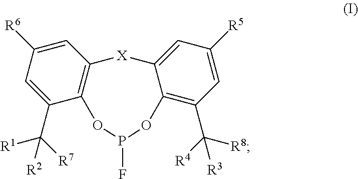

wherein:
X is —CH(R)—;
R is independently C₁₋₆alkyl, a 3- to 7-membered carbocycle, or a phenyl, where the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl;
R¹, R², R³, R⁴ are each independently C₁₋₆alkyl;
R⁵ and R⁶ are each

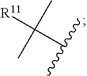

R¹¹is a C₁₋₆alkyl, a 3- to 6-membered carbocycle, or a phenyl, wherein the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl; and
R⁷ and R⁸ are each independently C₁₋₆alkyl, a 6- to 12-membered aryl, or a 3- to 6-membered carbocycle, wherein the 6- to 12-membered aryl and 3- to 6-membered carbocycle are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl.

E2. The fluorophosphite ligand of E1, wherein R is C₁₋₄alkyl or a 3- to 6-membered carbocycle.

E3. The fluorophosphite ligand of E1 or E2, wherein X is

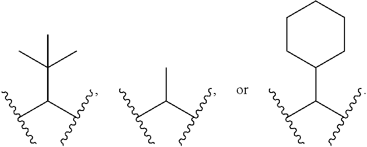

E4. The fluorophosphite ligand of E1-E3, wherein R¹, R², R³, and R⁴ are each C₁₋₄alkyl.

E5. The fluorophosphite ligand of E1-E4, wherein R¹, R², R³, and R⁴ are each —CH₃.

E6. The fluorophosphite ligand of any one of E1-E5, wherein R⁵ and R⁶ are each independently

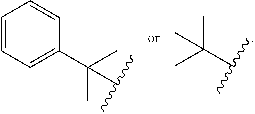

E7. The fluorophosphite ligand of any one of E1-E6, wherein R⁷ and R⁸ are each C₁₋₄alkyl or the unsubstituted or substituted phenyl.

E8. The fluorophosphite ligand of any one of E1-E7, wherein R⁷ and R⁸ are each —CH₃ or unsubstituted phenyl.

E9. The fluorophosphite ligand of any one of E1-E8, wherein the fluorophosphite ligand of formula (I) is a ligand of formula (Ia):

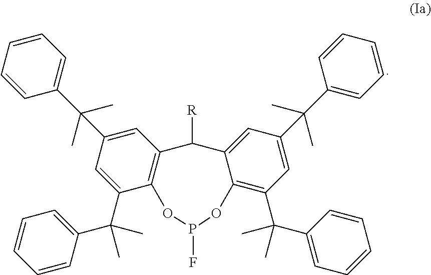

E10. The fluorophosphite ligand of any one of E1-E9, wherein the fluorophosphite ligand of formula (I) is a ligand of formula (Ib):

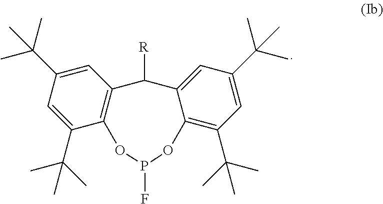

E11. The fluorophosphite ligand of any one of E1-E10, wherein the fluorophosphite ligand of formula (I) is

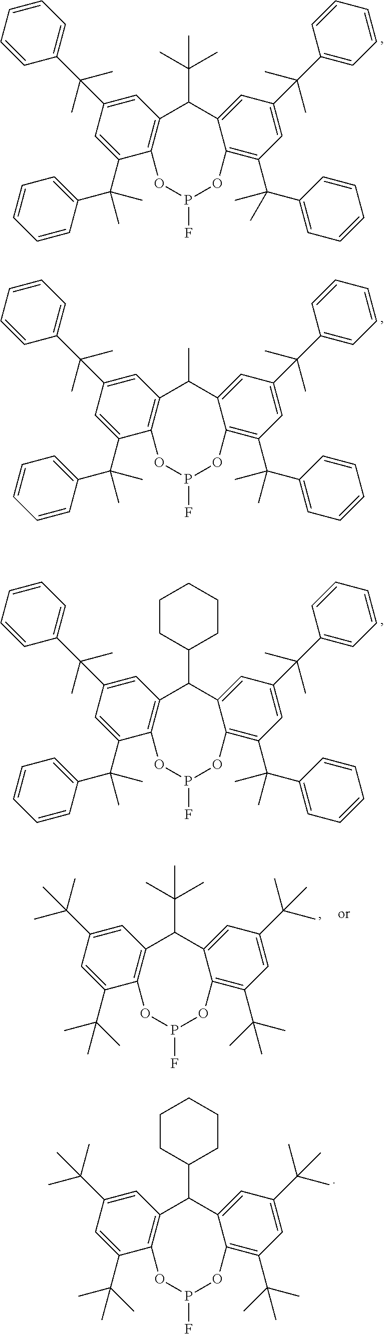

The fluorophosphite ligands of this disclosure may be prepared by any effective method. Various methods for preparing fluorophosphites are reported in the literature. For example, it has been found that fluorophosphites may be prepared by using a (benzyl)phenol starting material such as 2,2′-methylene bis(4,6-di(α,α-dimethylbenzyl)phenol) and following the procedures described in U.S. Pat. No. 4,912,155; Tullock et al., J. Org. Chem., 25, 2016 (1960); White et al., J. Am. Chem. Soc., 92, 7125 (1970); Meyer et al., Z. Naturforsch, Bi. Chem. Sci., 48, 659 (1993); or Puckette, in “Catalysis of Organic Reactions,” Edited by S. R. Schmidt, CRC Press (2006), pp. 31-38.

### 3. Catalyst Complex

The ratio of gram moles fluorophosphite ligand of formula (I) to gram atoms transition metal may vary over a wide range, e.g., gram mole fluorophosphite:gram atom transition metal ratio of 1:1 to 400:1. For rhodium-containing catalyst systems, the gram mole fluorophosphite:gram atom rhodium ratio in some aspects of the present disclosure is in the range of 1:1 to 200:1 with ratios in the range of 1:1 to 120:1.

In some aspects of the present disclosure, the ratio of Rh to the ligand of formula (I) ranges from 10:1 to 400:1.

In some aspects of the present disclosure, the ratio of Rh to the ligand of formula (I) ranges from 10:1 to 100:1.

In some aspects of the present disclosure, the ratio of Rh to the ligand of formula (I) ranges from 10:1 to 50:1.

The absolute concentration of rhodium in the reaction mixture or solution may vary from 1 mg/liter up to 5000 mg/liter or more. In some aspects of the present disclosure, the normal concentration of rhodium in the reaction solution may be in the range of 20 mg/liter (mg/L) to 300 mg/L. Concentrations of rhodium lower than this range may yield lower reaction rates with most olefin reactants and/or require reactor operating temperatures that are so high as to be detrimental to catalyst stability.

A catalyst of high activity may be obtained if all manipulations of the rhodium and fluorophosphite ligand components are carried out under an inert atmosphere, e.g., nitrogen, argon, and the like or if the catalyst is pre-activated (see IV. Experimental Examples, “Materials and Methods” for pre-activation conditions).

### B. Olefin

A wide variety of olefins may be used as the starting material for the methods disclosed herein. Specifically, olefins may include, but are not limited to, ethylene, propylene, butene, pentene, hexene, octene, styrene, non-conjugated dienes such as 1,5-hexadiene, and blends of these olefins. Furthermore, the olefin may be substituted with functional groups so long as they do not interfere with the hydroformylation reaction. Suitable substituents on the olefin may include any functional group that does not interfere with the hydroformylation reaction, such as, but not limited to, carboxylic acids and derivatives thereof such as esters and amides, alcohols, nitriles, and ethers. Examples of substituted olefins may include, but are not limited to, esters such as methyl acrylate or methyl oleate, alcohols such as allyl alcohol and 1-hydroxy-2,7-octadiene, and nitriles such as acrylonitrile.

In some aspects, the olefin may be a C₃₋₂₀alkene or a C₃₋₈cycloalkene. The olefin may be a C₃₋₁₀alkene. The olefin may be propylene, 1-octene, or 2-octene.

The amount of olefin present in the reaction mixture may vary. For example, relatively high-boiling olefins such as 1-octene may function both as the olefin reactant and the process solvent. In the hydroformylation of a gaseous olefin feedstock such as propylene, the partial pressures in the vapor space in the reactor are in the range of 1 psia to 500 psia. The rate of reaction may be favored by high concentrations of olefin in the reactor. In the hydroformylation of propylene, the partial pressure of propylene in some aspects of the present disclosure is greater than 20 psia, e.g., from 20 psia to 145 psia. In the case of ethylene hydroformylation, the partial pressure of ethylene in the reactor in some aspects of the present disclosure is greater than 2 psia.

### C. Hydroformylation Conditions

### 1. Hydroformylation Solvent

The hydroformylation solvent may be selected from a wide variety of compounds, mixture of compounds, or materials that are liquid at the pressure at which the process is being operated. Such compounds and materials include various alkanes, cycloalkanes, alkenes, cycloalkenes, carbocyclic aromatic compounds, alcohols, esters, ketones, acetals, ethers, and water. Examples of such solvents may include, but are not limited to, alkanes and cycloalkanes such as dodecane, decalin, octane, iso-octane mixtures, cyclohexane, cyclooctane, cyclododecane, methylcyclohexane; aromatic hydrocarbons such as benzene, toluene, xylene isomers, tetralin, cumene, alkyl-substituted aromatic compounds such as the isomers of diisopropylbenzene, triisopropylbenzene and tert-butylbenzene; alkenes and cycloalkenes such as 1,7-octadiene, dicyclopentadiene, 1,5-cyclooctadiene, 1-octene, 2-octene, 4-vinylcyclohexene, cyclohexene, 1,5,9-cyclododecatriene, 1-pentene; crude hydrocarbon mixtures such as naphtha, mineral oils and kerosene; and high-boiling esters such as 2,2,4-trimethyl-1,3-pentanediol diisobutyrate. The aldehyde product of the hydroformylation process also may be used.

The solvent may include the higher boiling by-products that are naturally formed during the process of the hydroformylation reaction and the subsequent steps, e.g., distillations, that may be required for aldehyde product isolation. The main criterion for the solvent is that it dissolves the catalyst and olefin substrate and does not act as a poison to the catalyst. Some examples of solvents for the production of volatile aldehydes, e.g., butyraldehydes, may be those that are sufficiently high boiling to remain, for the most part, in a gas sparged reactor. Some examples of solvents and solvent combinations that are useful in the production of less volatile and nonvolatile aldehyde products include 1-methyl-2-pyrrolidinone, dimethyl formamide, perfluorinated solvents such as perfluoro-kerosene, sulfolane, water, and high boiling hydrocarbon liquids as well as combinations of these solvents. Non-hydroxylic compounds, in general, and hydrocarbons, in particular, may be used advantageously as the hydroformylation solvent since their use may minimize decomposition of the fluorophosphite ligands. In some aspects of the present disclosure, the hydroformylation solvent is toluene.

### 2. Temperature

The reaction conditions for the process of the present disclosure may include conventional hydroformylation conditions. The process may be carried out at temperatures in the range of 50° C. to 135° C. In some aspects of the present disclosure, reaction temperatures may range from 60° C. to 130° C. The temperature in the feed may be selected according to the desired linear:branched aldehyde ratio (l/b). In some aspects of the present disclosure, the reaction temperature ranges from 95° C. to 130° C. In other aspects, the reaction temperature ranges from 60° C. to 100° C. In some aspects of the present disclosure, the temperature ranges from 100° C. to 120° C. In other aspects, the temperature ranges from 75° C. to 85° C.

### 3. Partial Hydrogen Pressure (P_{H2}) and Partial Carbon Monoxide Pressure (P_{CO})

The total reaction pressure may range from ambient or atmospheric pressure up to 1000 psia. The hydrogen:carbon monoxide mole ratio in the reactor likewise may vary considerably ranging from 10:1 to 1:10, and the sum of the absolute partial pressures of hydrogen and carbon monoxide may range from 5 psia to 500 psia. The partial pressures of the ratio of the hydrogen to carbon monoxide in the feed may be selected according to the linear:branched aldehyde ratio (l/b) desired. Generally, the partial pressure of hydrogen (P_{H2}) and carbon monoxide (P_{CO}) in the reactor may be maintained within the range of 5 psia to 350 psia for each gas. The partial pressure of carbon monoxide (P_{CO}) in the reactor may be maintained within the range of 5 psia to 350 psia and may be varied independently of the hydrogen partial pressure (P_{H2}). The partial pressure of hydrogen (P_{H2}) in the reactor may be maintained within the range of 5 psia to 350 psia and may be varied independently of the carbon monoxide partial pressure (P_{CO}). The molar ratio of hydrogen to carbon monoxide may be varied widely within these partial pressure ranges for the hydrogen and carbon monoxide. The ratios of hydrogen-to-carbon monoxide and the partial pressure of each in the synthesis gas (syngas-carbon monoxide and hydrogen) may be readily adjusted by the addition of either hydrogen or carbon monoxide to the syngas stream. With the fluorophosphite ligands described herein, the ratio of linear to branched (l/b) products may be adjusted by changing the partial pressures of the carbon monoxide in the reactor.

For example, in some aspects of the present disclosure, the P_{CO} may range from 110 psia to 350 psia and the P_{H2} may range from 20 psia to 150 psia. In other aspects, the P_{CO} may range from 5 psia to 150 psia and the P_{H2} may range from 100 psia to 350 psia.

### 4. Reactor Design

Any of the known hydroformylation reactor designs or configurations such as overflow reactors and vapor take-off reactors may be used in carrying out the process provided by the present disclosure. Thus, a gas-sparged, vapor take-off reactor design may be used. In this mode of operation, the catalyst, which is dissolved in a high boiling organic solvent under pressure, does not leave the reaction zone while the aldehyde product is taken overhead by the unreacted gases. The overhead gases then are chilled in a vapor/liquid separator to liquefy the aldehyde product and the gases may be recycled to the reactor. The liquid product may be let down to atmospheric pressure for separation and purification by conventional techniques. The process also may be performed in a batchwise manner by contacting the olefin, hydrogen and carbon monoxide with the present catalyst in an autoclave.

A reactor design where catalyst and feedstock are pumped into a reactor and allowed to overflow with product aldehyde, i.e., liquid overflow reactor design, may be used. For example, high boiling aldehyde products such as nonyl aldehydes may be prepared in a continuous manner with the aldehyde product being removed from the reactor zone as a liquid in combination with the catalyst. The aldehyde product may be separated from the catalyst by conventional means such as by distillation or extraction, and the catalyst then recycled back to the reactor. Water soluble aldehyde products, such as hydroxy butyraldehyde products obtained by the hydroformylation of allyl alcohol, may be separated from the catalyst by extraction techniques. A trickle-bed reactor design is also suitable for this process. It will be apparent to those skilled in the art that other reactor schemes may be used with the processes of this disclosure.

### 5. Modulation of Linear:Branched Aldehyde Ratio (l/b)

The temperature, partial pressure of hydrogen (P_{H2}), and partial carbon monoxide (P_{CO}) in the feed may be selected according to the desired linear:branched aldehyde ratio (l/b).

### a. Branched Selective (l/b<1)

In some aspects, contacting the olefin with H₂ and CO in the presence of the catalyst solution at a temperature ranging from 95° C. to 130° C., a P_{CO} ranging from 110 psia to 350 psia, and a P_{H2} ranging from 20 psia to 150 psia, produces l/b of less than 1.0. In some aspects, contacting the olefin with H₂ and CO in the presence of the catalyst solution at a temperature ranging from 100° C. to 120° C., a P_{CO} ranging from 130 psia to 230 psia, and a P_{H2} ranging from 30 psia to 130 psia, produces l/b of less than 0.90. In other aspects, contacting the olefin with H₂ and CO in the presence of the catalyst solution at a temperature ranging from 100° C. to 120° C., a P_{CO}ranging from 200 psia to 300 psia, and a P_{H2} ranging from 20 psia to 50 psia, produces l/b of less than 0.60.

### b. Linear Selective (l/b>1)

In some aspects, contacting the olefin with H₂ and CO in the presence of the catalyst solution at a temperature ranging from 60° C. to 100° C., a P_{CO} ranging from 5 psia to 150 psia, and a P_{H2} ranging from 100 psia to 350 psia, produces l/b of greater than 1.0. In some aspects, contacting the olefin with H₂ and CO in the presence of the catalyst solution at a temperature ranging from 75° C. to 85° C., a P_{CO} ranging from 5 psia to 10 psia, and a P_{H2} ranging from 200 psia to 300 psia, produces l/b of greater than 1.6. In some aspects, contacting the olefin with H₂ and CO in the presence of the catalyst solution at a temperature ranging from 75° C. to 85° C., a P_{CO} ranging from 50 psia to 150 psia, and a P_{H2} ranging from 110 psia to 210 psia, produces a l/b of greater than 1.6.

### IV. Experimental Examples

The foregoing may be better understood by reference to the following examples, which are presented for purposes of illustration and are not intended to limit the scope of the technology.

### Abbreviations

g is gram(s);
mg is milligram(s);
L is liter(s);
mL is milliliter(s);
μL is microliter(s);
m is meter(s);
mm is millimeter(s);
μm is micrometer(s);
mol or mol. is mole(s);
mmol or mmol. is millimole(s);
M is mole(s) per liter;
mM is millimole(s) per liter;
J is joule(s);
kJ is kilojoule(s);
t is time;
T is temperature;
s or sec is second(s);
h or hr is hour(s);
min or min. is minute(s);
rt, RT, or r.t. is room temperature;
eq, eq., or equiv is equivalent(s);
sat. is saturated;
solv. is solvent;
OD is outer diameter;
ID is inner diameter;
Hydroform. is hydroformylation;
ald is aldehyde;
MePh is toluene;
MeOH is methanol;
DCM is dichloromethane;
t-BuOH is tert-butanol;
EtMgBr is ethyl magnesium bromide;
TOF is turn over frequency;
TOS is time on stream;
GC-MS is gas chromatography mass spectrometry;
HRMS is high-resolution mass spectrometry;
EI is electron ionization;
acac is acetylacetonate;
t_{R} is residence time;
psia is pounds per square inch absolute;
P_{CO} is pressure of carbon monoxide;
P_{H2} is pressure of hydrogen; and
l/b is linear to branched.

### General Materials and Methods

### A. Materials

Toluene (99.8% Anhydrous), 1-octene, nonanal (95%), butyraldehyde (99.5%), isobutraldehyde (+99%), and 1,3,5-trimethoxybenzene, 2,4-bis(α,α-dimethylbenzyl)phenol, trioctylphosphine, and ethylmagnesium bromide in ether (3.0 M) were purchased from Sigma Aldrich. 2-Methyl octanal was purchased from BOC chemicals. Dicarbonyl 2,4-pentanedionato rhodium (I) 97% and 75% trimethylacetaldehyde in t-BuOH were purchased from Alfa Aesar. 2-Octene (mixture of cis+trans isomer) (+98%) was purchased from TCI. Deuterated DCM was purchased from Fisher Scientific. Carbon monoxide, hydrogen, and nitrogen were purchased from Airgas at 99.9% purity. Propylene was purchased from Airgas at 99.95% purity. 1-octene and 2-octene were purified over silica gel columns before use. Octene isomers were degassed with argon before use.

### B. Synthesis of Fluorophosphite Ligands

The preparation of cyclic monofluorophosphite ligand L was accomplished via previously described methods as shown in Scheme A below.

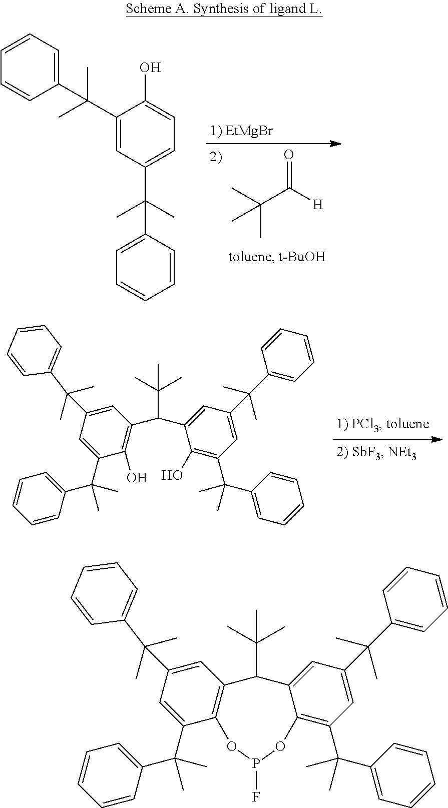

Ligands analogous to L may be prepared similarly using the appropriate starting materials.

### Synthesis of 12-(tert-butyl)-6-fluoro-2,4,8,10-tetrakis(2-phenylpropan-2-yl)-12H-dibenzo[d,g][1,3,2]dioxaphosphocine (L)

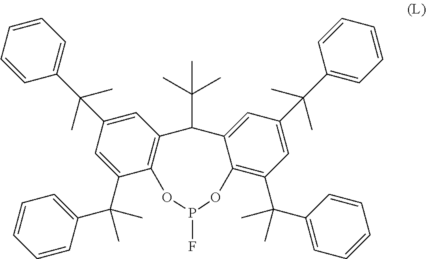 [SMILES: CC(C)(c1ccccc1)c1cc2c(c(C(C)(C)c3ccccc3)c1)OP(F)Oc1c(cc(C(C)(C)c3ccccc3)cc1C(C)(C)c1ccccc1)C2C(C)(C)C]

### i. 6,6′-(2,2-Dimethylpropane-1,1-diyl)bis(2,4-bis(2-phenylpropan-2-yl)phenol)

2,4-Bis(α,α-dimethylbenzyl)phenol (850 g, 2.6 moles) and 1.8 L of toluene were combined under nitrogen in a 5-L, 3-neck round bottom flask equipped with boiling stones, a Dean-Stark trap, and a glycol-cooled condenser. The reaction mixture was heated to reflux for a period of time sufficient to collect 200 mL of liquid in the Dean-Stark trap, after which the flask was allowed to cool naturally to ambient temperature. The Dean-Stark trap was replaced with an addition funnel containing 800 mL of 3 Methylmagnesium bromide (2.4 moles) in ether followed by dropwise addition of the Grignard reagent. Once complete, the mixture was allowed to cool to ambient temperature for 1 h, after which a clean addition funnel containing 165 g of 75% trimethylacetaldehyde/t-BuOH (1.4 moles) was attached to the flask and added. After the removal each addition funnel, a clean reflux condenser was attached, and the reaction mixture was heated to reflux for 2 days (color is initially a bright green that slowly transitions to brown). After cooling to ambient temperature, a magnetic stir bar was added, and the reaction was quenched with slow addition of 1.25 L of 2 M HCl followed by rapid stirring overnight. The organic phase was then washed with 2× 500 mL of deionized water followed by slow evaporation of solvent with nitrogen gas flow. The crude solid was transferred to a 4 L beaker containing MeOH (1 L) and stirred mechanically until a white powder precipitates, after which the slurry was heated to a boil for 5 min, then allowed to cool naturally to ambient temperature. The white precipitate was the filtered and washed with 2×500 mL of MeOH, after which the final product was dried in vacuo to give 699 grams (80% isolated yield) of white powder.

### ii. 12-(tert-Butyl)-6-fluoro-2,4,8,10-tetrakis(2-phenylpropan-2-yl)-12H-dibenzo[d,g][1,3,2]dioxaphosphocine (L)

Under a nitrogen atmosphere, 6,6′-(2,2-dimethylpropane-1,1-diyl)bis(2,4-bis(2-phenylpropan-2-yl)phenol) was dissolved in a triethylamine in toluene solution at a ratio of 1.4 g phenol:1 mL triethylamine:4.3 mL toluene and added to a cooled (5° C.) solution of PCl₃ in toluene (0.09 mL PCl₃:1 mL toluene) over a time period of 1.25 h. At the end of the addition, the slurry was stirred for additional 15 min then warmed to room temperature. After stirring overnight at room temperature, SbF₃ was added to the mixture, and the temperature was raised to 85° C. and kept at 85° C. stirring for 6.5 h. After cooling, the toluene layer was separated and purified by column chromatography. ¹H NMR (600 MHZ, CD₂Cl₂): δ 8.14-6.64 (m, 24H, ArH), 4.21 (s, 1H, bridge C—H), 2.09-1.28 (m, 24H, 4×C(CH₃)₂), 0.74 (s, 9H, bridge C(CH₃)₃). ¹³C NMR (151 MHz, CD₂Cl₂): 150.61-150.40 C(1), 149.07-148.94 C(2), 146.40-145.80 C(3), 138.84-122.80 C(4), 46.27-45.41 C(5), 42.80-41.93 C(6), 35.96-35.82 C(7), 33.26-27.88 C(8). ³¹P NMR (243 MHZ, CD₂Cl₂): δ 130.18 (d, J=1305.7 Hz), 127.47 (d, J=1296.3 Hz), 98.01 (d, J=1269.1 Hz). ¹⁹F NMR (564 MHz, CD₂Cl₂): δ −67.25-−70.30 (m), −69.55 (d, J=1269.9 Hz), −70.40 (d, J=1305.8 Hz). Three Isomers of L are observed by NMR:

| Isomer | % (³¹P) | % (¹⁹F) |
|---|---|---|
| A | 76 | 75.9 |
| B | 18.5 | 19.1 |
| C | 5.5 | 5 |

HRMS: (EI) m/z: [M]⁺ Calcd for C₅₃H₅₈FO₂P: 776.42; Found: 776.42

### C. Batch Reactions

The desired amount of ligand L was weighed in a glass container and the corresponding amount of dicarbonyl 2,4-pentanedionato rhodium (I) was added from a 1 mg/mL stock solution in toluene. The solution was diluted with toluene to [Rh]=0.25 mM. The olefin (1- or 2-octene) was added to the solution at a concentration of 0.5 M and 2 mL of the final solution was transferred to a glass vial. No octene was added in the propylene hydroformylation experiment. The glass vial containing the liquid mixture and a magnetic stir bar was placed in a Buchiglas Tinyclave (35 mL)—except for experiments with P_{CO}=6 psia which were conducted in a Buchiglas Miniclave (280 mL)—under inert atmosphere. The autoclave was connected to the gas supply manifold and the lines were purged three times with nitrogen prior to each experiment. The autoclave was then opened and purged an additional three times with nitrogen and two times with hydrogen. Carbon monoxide and hydrogen pressures were then sequentially introduced into the autoclave. The autoclave was sealed under pressure. The manifold was then purged with nitrogen and the autoclave was disconnected and placed in an oil bath on a hot plate. Upon reaction completion, the autoclave was removed from the oil bath and cooled in a water bath until it reached room temperature. After the autoclave was cooled down to the room temperature it was reconnected to the gas manifold. The autoclave was vented through the manifold and purged three times with nitrogen before opening. Aliquots were taken from the liquid mixture inside the vial and diluted with toluene for analysis.

### D. Flow Reactions

Stainless steel syringes (8 mL) containing 0.5 M 1-octene in toluene, 0.25 mM Rh in toluene, and 10:1 L:Rh were prepared under inert atmosphere and then connected to the stainless steel flow reactor coil (⅛″ outer diameter, OD,× 1/16″ inner diameter, ID) with a 40-cm long fluorinated ethylene propylene tubing (Microsolv, 1/16″ OD×0.02″ ID), and 1/16″ OD nuts and ferrules (IDEX H&S). Liquid flowrates were controlled by Harvard PHD ULTRA syringe pumps and gas flowrates were controlled by individual Bronkhorst mass flow controllers (EL-Flow® Select MFCs). The flow reactor temperature was controlled through a hotplate and oil bath with a temperature probe. The flow reactor pressure was controlled with a nitrogen pressure connected to the control port of an Equilibar backpressure regulator integrated at the outlet of the flow reactor coil. The flow reactor effluent was passed through a 10-way selector valve (VICI, EUHB) and directed to a custom-designed waste collection chamber equipped with an exhaust line for CO and H₂. After changing reaction conditions, the flow reactor was allowed to stabilize for twice the length of the residence time for a given reaction condition, before a sample was taken by directing the selector valve towards a collection vial for 30 min. Following the sample collection, the flow reactor effluent was directed to the waste vial during the transient period of the next hydroformylation reaction condition.

### E. Catalyst Pre-Activation with the Cyclic Monofluorophosphite Ligand

The desired amount of ligand L was weighed in a glass vial and the corresponding amount of dicarbonyl 2,4-pentanedionato rhodium (I) was added from a 1 mg/mL stock solution in toluene. The solution was diluted with toluene to the target Rh concentration. The glass vial equipped with a magnetic stir bar was then placed in a Buchiglas Miniclave (280 mL) under inert atmosphere. The autoclave was connected to the gas supply manifold and the lines were purged three times with nitrogen. The autoclave was then opened and purged an additional three times with nitrogen and two times with hydrogen. Carbon monoxide and hydrogen pressures were then introduced sequentially each at 75 psia (total pressure of 150 psia). The autoclave was sealed under pressure. The gas manifold was then purged with nitrogen and the autoclave was disconnected and placed in an oil bath at 85° C. After 1 h, the autoclave was taken out of the oil bath and cooled in a water bath until it reached room temperature. After the autoclave was cooled down to the room temperature, it was then reconnected to the gas manifold. The autoclave was vented through the gas manifold and purged three times with nitrogen before opening.

### F. Characterization-Product Analysis by Gas Chromatography (GC)

Aliquots of the reaction samples (100 μL for each sample) were collected from either batch or flow reactor and diluted with 1 mL of toluene and 100 L of 0.05 M 1,3,5-trimethoxybenzene in toluene as an internal calibration standard. 1 μL of GC mixture was injected into Shimadzu GCMS-2010 with a Zebron ZB-5MSi column 30 m×0.25 mm×0.25 μm.

### 1. GC Method

7 min at 40° C. then 20° C./min to 5 min at 85° C. and 20° C./min to 210° C. Product calibrations were performed on 1-octene, cis-2-octene, trans-2-octene, trans-3-octene, trans-4-octene, cis-4-octene, nonanal, 2-methyl octanal, isobutyraldehyde, and n-butyraldehyde. The ratio of 2-propylhexanal/2-ethylheptanal/2-methyl octanal/nonanal in the hydroformylation product was calculated from peak area ratio and verified by NMR.

### 2. NMR Verification

30 mg of L was dissolved in 1 mL of deuterated DCM and the ¹H, ¹⁹F, and ³¹P spectra were collected with a Bruker Avance NEO 600 MHz NMR.

### EXAMPLE 1

### Batch vs. Flow Hydroformylation of 1-Octene

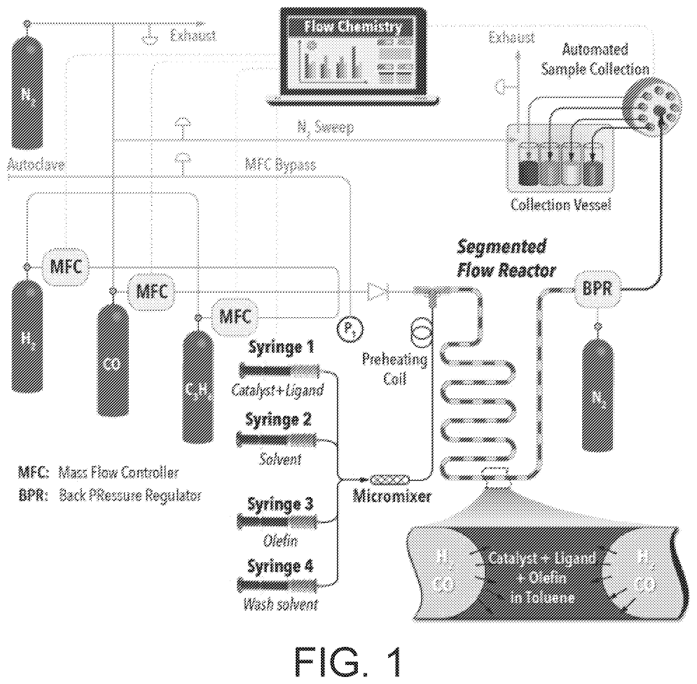

The hydroformylation of 1-octene in toluene was performed in a continuous flow and a batch reactor at four different temperatures (65° C., 75° C., 95° C., and 110° C.). The developed flow chemistry platform, utilizing a continuous segmented flow reactor, for accelerated fundamental studies of hydroformylation of olefins is illustrated FIG. 1. The rhodium dicarbonyl acetylacetone catalyst (Rh(CO)₂acac) concentration was 0.25 mM or 0.05 mol % of 1-octene and L was added at a 10:1 L:Rh ratio. The initial partial pressures of CO(P_{CO} ) and H₂ (P_{H2} ) were each set at 100 psia. The gas to liquid volumetric ratio was 3:1 in flow and 17:1 in batch. The reaction time in flow was varied from 5 min to 50 min. The batch reaction time was set at 2 h. Product analysis showed that four aldehyde products (l, b1, b2, and b3) were formed in addition to 2-, 3-, and 4-octene isomers and n-octane (Scheme 1).

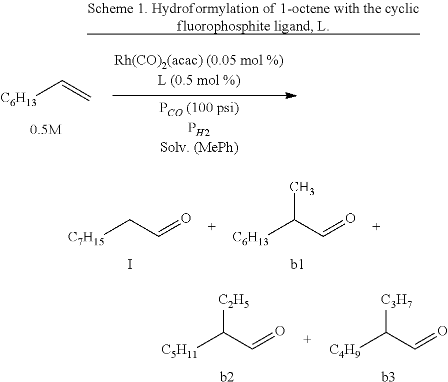

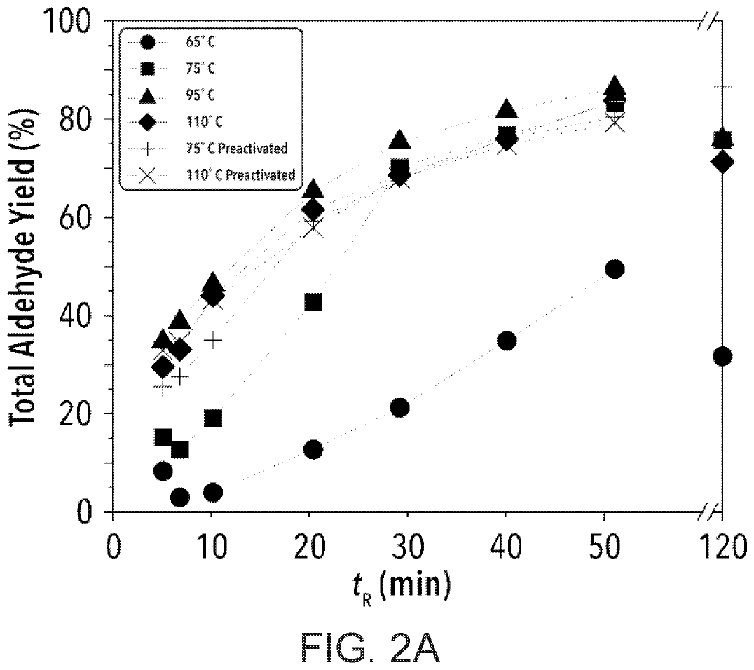

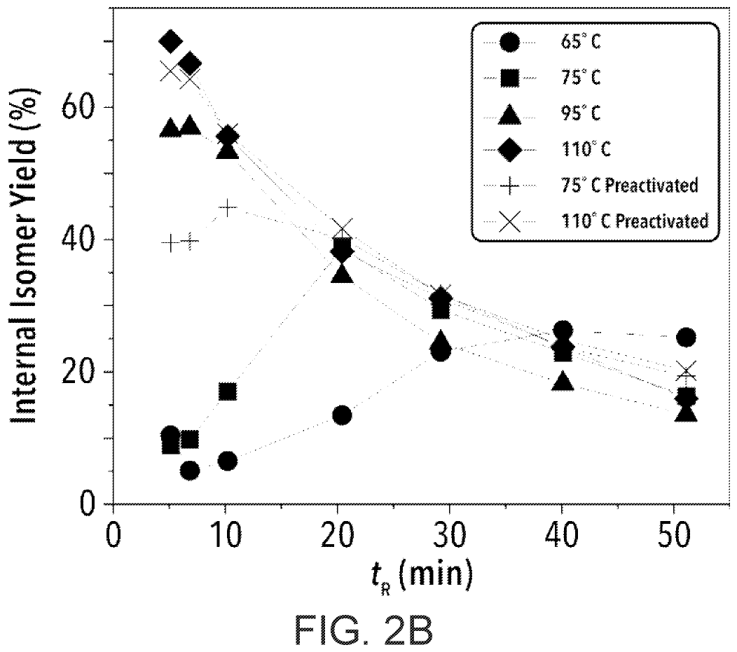

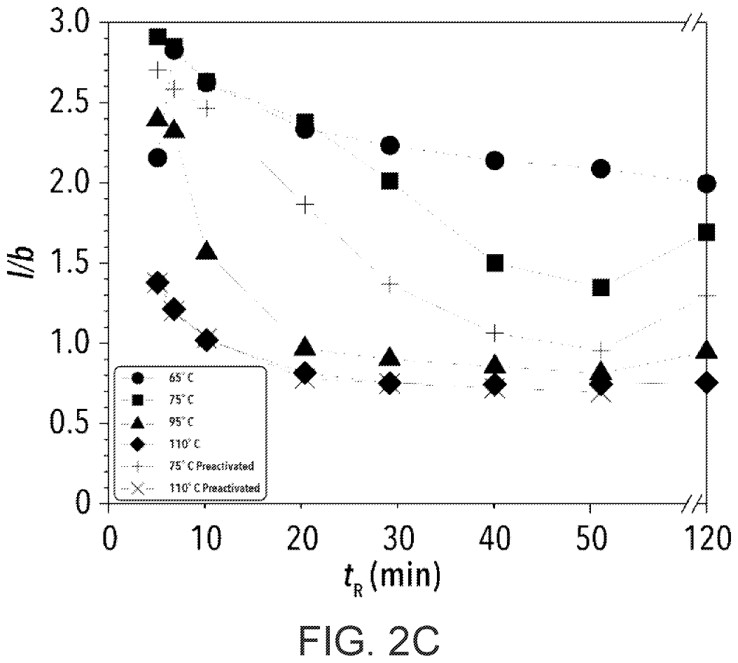

In the first set of experiments, the difference in activity and selectivity between reactions performed under batch versus flow conditions was investigated. The total aldehyde yield, internal isomer yield, and aldehyde regioselectivity (i.e., l/b=l/(b1+b2+b3)) were monitored. Data was collected at different reaction (i.e., residence) times, t_{R}, in the flow reactor and at a 2 h reaction time in the batch reactor. These data are presented in FIG. 2A-2C and Table 1.

TABLE 1
1-octene consumption and hydroformylation, isomerization, and hydrogenation
yield in flow. P_{CO} = 100 psia and P_{H2} = 100 psia.

| | t_{R} (min) | | | | | | |
|---|---|---|---|---|---|---|---|
| | 5.1 | 6.82 | 10.2 | 20.4 | 29.2 | 40.1 | 51.1 |
| T (° C.) | 110 | 110 | 110 | 110 | 110 | 110 | 110 |
| l/b | 1.38 | 1.21 | 1.02 | 0.81 | 0.75 | 0.74 | 0.75 |
| Aldehyde yield (%) | 30 | 33 | 44 | 62 | 69 | 76 | 84 |
| Isomerization yield (%) | 70 | 67 | 56 | 38 | 31 | 24 | 16 |
| Hydrogenation yield (max) (%) | 0.00 | 0.00 | 0.00 | 0.00 | 0.00 | 0.00 | 0.00 |
| % Unreacted 1-Octene | 0.51 | 0.28 | 0.32 | 0.21 | 0.25 | 0.22 | 0.21 |
| TOF (mol ald · mol Rh⁻¹ · h⁻¹) | 6937 | 5818 | 5175 | 3617 | 2815 | 2271 | 1965 |
| T (° C.) | 95 | 95 | 95 | 95 | 95 | 95 | 95 |
| l/b | 2.39 | 2.32 | 1.56 | 0.97 | 0.90 | 0.86 | 0.81 |
| Aldehyde yield (%) | 35 | 39 | 46 | 65 | 75 | 82 | 86 |
| Isomerization yield (%) | 57 | 57 | 53 | 34 | 24 | 18 | 14 |
| Hydrogenation yield (max) (%) | 0.00 | 0.00 | 0.00 | 0.00 | 0.00 | 0.00 | 0.00 |
| % Unreacted 1-Octene | 8.7 | 4.4 | 0.4 | 0.3 | 0.3 | 0.1 | 0.1 |
| TOF (mol ald · mol Rh⁻¹ · h⁻¹) | 8162 | 6792 | 5448 | 3834 | 3092 | 2439 | 2025 |
| T (° C.) | 75 | 75 | 75 | 75 | 75 | 75 | 75 |
| l/b | 2.91 | 2.85 | 2.63 | 2.38 | 2.01 | 1.50 | 1.35 |
| Aldehdye yield (%) | 15 | 13 | 19 | 43 | 70 | 77 | 83 |
| Isomerization yield (%) | 9 | 10 | 17 | 39 | 29 | 23 | 16 |
| Hydrogenation yield (max) (%) | 0.00 | 0.00 | 0.00 | 0.00 | 0.00 | 0.00 | 0.00 |
| % Unreacted 1-Octene | 76 | 77 | 64 | 18 | 0.5 | 0.3 | 0.3 |
| TOF (mol ald · mol Rh⁻¹ · h⁻¹) | 3591 | 2245 | 2256 | 2513 | 2877 | 2293 | 1953 |
| T (° C.) | 65 | 65 | 65 | 65 | 65 | 65 | 65 |
| l/b | 2.16 | 2.83 | 2.62 | 2.33 | 2.23 | 2.14 | 2.09 |
| Aldehyde yield (%) | 8 | 3 | 4 | 13 | 21 | 35 | 49 |
| Isomerization yield (%) | 10 | 5 | 7 | 13 | 23 | 26 | 25 |
| Hydrogenation yield (max) (%) | 0.00 | 0.00 | 0.00 | 0.00 | 0.00 | 0.00 | 0.00 |
| % Unreacted 1-Octene | 81 | 92 | 89 | 74 | 56 | 39 | 25 |
| TOF (mol ald · mol Rh⁻¹ · h⁻¹) | 1957 | 524 | 470 | 749 | 872 | 1042 | 1160 |
| | Pre-activated | | | | | | |
| T (° C.) | 75 | 75 | 75 | 75 | 75 | 75 | 75 |
| | 2.70 | 2.58 | 2.47 | 1.86 | 1.37 | 1.06 | 0.96 |
| Aldehyde yield (%) | 25.51 | 27.58 | 35.05 | 59.25 | 67.89 | 75.96 | 80.45 |
| Isomerization yield (%) | 39.52 | 39.78 | 44.81 | 40.21 | 31.79 | 23.75 | 19.37 |
| Hydrogenation yield (max) (%) | 0.00 | 0.00 | 0.00 | 0.00 | 0.00 | 0.00 | 0.00 |
| % Unreacted 1-Octene | 34.96 | 32.64 | 20.14 | 0.54 | 0.32 | 0.29 | 0.18 |
| TOF (mol ald · mol Rh⁻¹ · h⁻¹) | 5994 | 4846 | 4118 | 3480 | 2786 | 2270 | 1886 |
| | Pre-activated | | | | | | |
| T (° C.) | 110 | 110 | 110 | 110 | 110 | 110 | 110 |
| l/b | 1.38 | 1.2 | 1.03 | 0.78 | 0.75 | 0.72 | 0.69 |
| Aldehyde yield (%) | 33 | 35 | 43 | 58 | 68 | 75 | 79 |
| Isomerization yield (%) | 65 | 64 | 56 | 42 | 32 | 25 | 20 |
| Hydrogenation yield (max) (%) | 0 | 0 | 0 | 0 | 0 | 0 | 0 |
| % Unreacted 1-Octene | 1.64 | 1.2 | 0.7 | 0.4 | 0.4 | 0.3 | 0.5 |
| TOF (mol ald · mol Rh⁻¹ · h⁻¹) | 7733 | 6074 | 5088 | 3401 | 2788 | 2235 | 1860 |

For these experiments, the maximum aldehyde yield was observed at 95° C. for both the batch and flow reactor systems (FIG. 2A). The catalyst reactivity appeared to decrease as the reaction temperature was decreased from 75° C. to 65° C., with only 75% 1-octene conversion measured at 50 min. The total aldehyde yield did not change as the reaction temperature was increased from 75° C. to 110° C. at t_{R}>30 min in flow. This trend was also observed for the 2 h batch experiments. The aldehyde yields of the 2 h batch reactions were obtained in the flow reactor in only 30 min reaction time at 95° C. and 110° C., and in 40 min at 65° C. to 75° C. This accelerated kinetics is rationalized by the enhanced heat and mass transfer rates of the continuous flow reactor (process intensification) compared with the batch reactor.

S-shaped yield vs. time curves were observed at 75° C. and 65° C. with a decrease in reactivity between 5 min and 7 min (FIG. 2A). This trend is likely attributed to the catalyst activation period. The experiments conducted at 75° C. were repeated with a pre-activated catalyst and the ligand mixture. Pre-activation increased the total aldehyde yield at the low t_{R} and eliminated the S-shape behavior associated with the catalyst activation period in flow (FIG. 2A).

The reaction temperature appeared to have a greater effect on accelerating the olefin isomerization than hydroformylation (FIG. 2B). Increasing the reaction temperature from 75° C. to 95° C. increased the yield of internal octenes from 10% to 55% in 5 min (FIG. 2B). Full conversion of 1-octene was achieved in the flow reactor at 6 min, 12 min, and 37 min at reaction temperatures 110° C., 95° C., and 75° C. respectively (see Table 1 for 1-octene conversion and isomerization). Rapid olefin isomerization was also observed with the catalyst pre-activation conditions, as demonstrated by the increase in the yield of internal octene isomers from 10% to 40% at 75° C. At 110° C., the olefin isomerization yield ranged from 65% to 70%. Further, the olefin isomerization yield did not change noticeably with the catalyst pre-activation. The olefin isomerization yield decreased for longer reaction times (FIG. 2B).

Despite achieving similar total aldehyde yields at 30 min for reaction temperatures of 75° C., 95° C., and 110° C., the l/b ratio decreased from 1.5 to 0.75 in flow, and from 1.7 to 0.76 in batch, as the reaction temperature increased (FIG. 2C). With no change to the temperature, the l/b decreased upon catalyst pre-activation both in batch and flow reactors. The catalyst pre-activation conditions did not impact the aldehyde yield when the hydroformylation was performed at 110° C., indicating a rapid in-situ catalyst pre-activation at this temperature. Based on these experimental flow and batch 1-octene hydroformylation results with L, shown in FIGS. 2A-2C, it appears that the aldehyde regioselectivity may be tuned from 0.75 to 2.5 by adjusting the reaction temperature and implementing pre-activated catalyst.

### EXAMPLE 2

### Mechanistic Studies of 1-Octene Hydroformylation in Flow

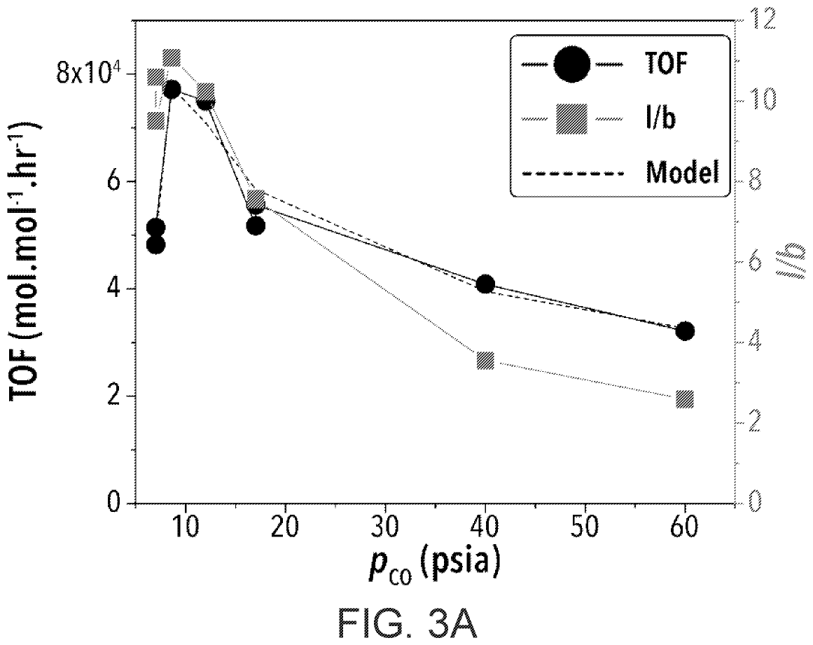

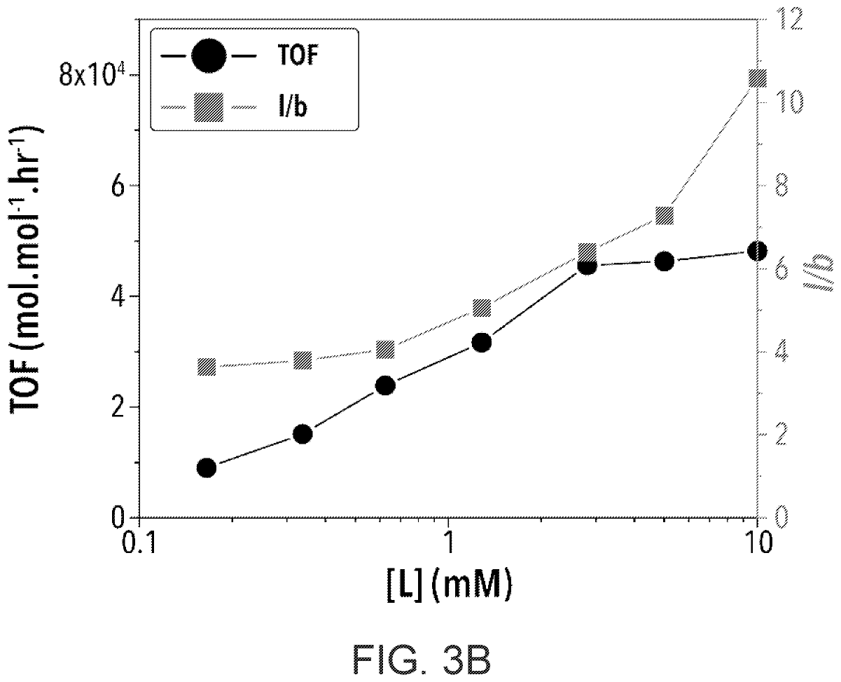

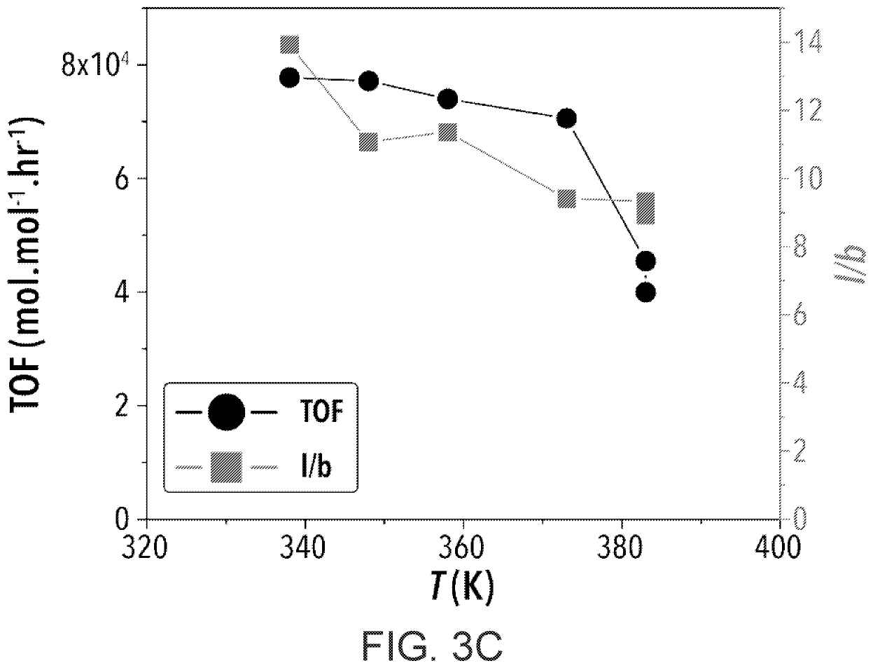

To understand the effect of the different reaction parameters on the aldehyde regioselectivity and the differences between batch and flow, the effects of reaction parameters were systematically investigated. The reaction parameters explored were the CO pressure (P_{CO}), the ligand concentration [L], and the temperature for 1-octene hydroformylation at fractional conversion. Under the experimental conditions shown in FIGS. 3A-3C, only l and b1 aldehydes were formed, and the total aldehyde yield was less than 30%. The CO consumption remained less than 15% by keeping the t_{R} at 21 min. The effect of P_{CO} was investigated at 75° C. with a pre-activated catalyst at Rh loading of 1 mmol %. The L:Rh ratio was increased to 2000:1 ([L]=10 mM). The hydroformylation TOF sharply increased as P_{CO} decreased from 60 psia to 12 psia (FIG. 3A). At P_{CO} below 12 psia, a decrease in the TOF was observed, indicating that the reaction at P_{CO}<12 psia is limited by CO. Further, the l/b ratio increased with the increasing TOF to reach 11.1 (91.7% linear) at TOF=75,000 mol ald·mol Rh⁻¹·h⁻¹.

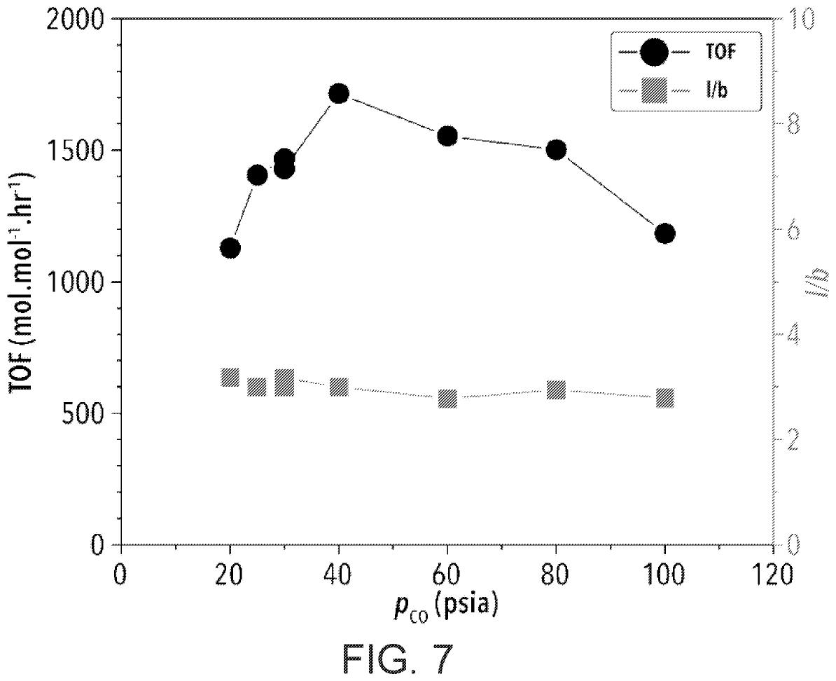

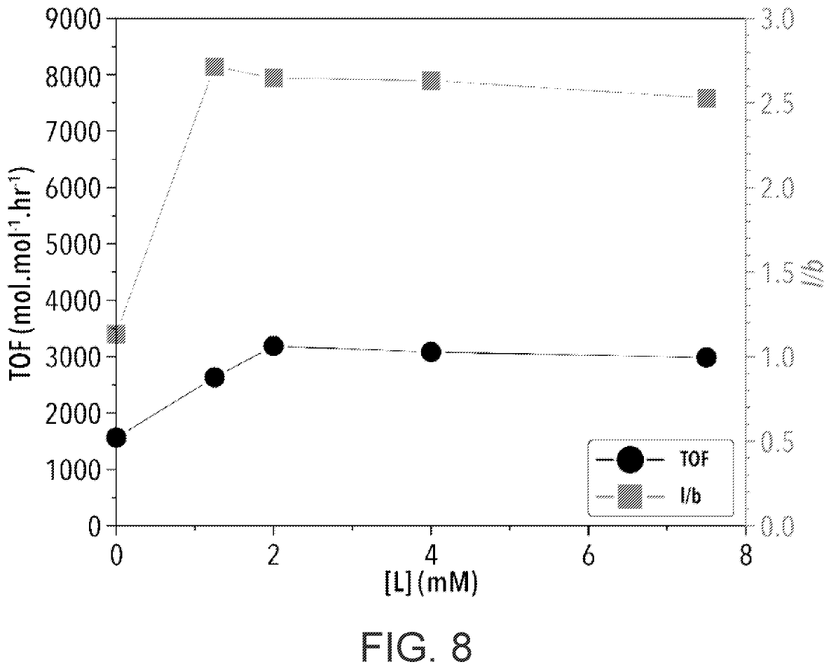

Hydroformylation with a pre-activated Rh catalyst in the presence of L at variable [L] and at P_{CO}=7 psia demonstrated a monotonic increase in the l/b selectivity with [L], shown in FIG. 3B. The increase in l/b suggests a change in the Rh coordination sphere as L:Rh increases up to 2000:1. The activation effect of Lon the Rh catalyst is also indicated by the 4.5 folds increase in the TOF as [L] was increased from 0.17 mM to 2.8 mM. Beyond this ligand concentration, no increase in the TOF was observed and TOF reached a maximum of 45,000 mol ald·mol Rh⁻¹·h⁻¹. A control reaction was run without the addition of L, and the highest activity achieved with the pre-activated Rh(CO)₂acac in flow was 1,700 mol ald·mol Rh⁻¹·h⁻¹ (FIG. 7). This observation indicates that 28-fold increase in the TOF is enabled by L at concentrations equal or higher than 2.8 mM. Ligand concentration screening experiments of L at higher P_{CO} and reaction temperature (135 psia and 80° C.) resulted in a lower induction in the TOF and l/b (FIG. 8), indicating that the ligand effect on activity and selectivity is amplified at low P_{CO}.

The graph depicted in FIG. 3C illustrates the effect of reaction temperature on the TOF and aldehyde regioselectivity at a constant P_{CO} and [L]. The data suggests that the TOF and l/b decreases with increasing the reaction temperature. At 65° C., the TOF was 77,700 mol ald·mol Rh⁻¹·h⁻¹, and the l/b was 13.9. For all hydroformylation reaction conditions tested in FIGS. 3A-3C only l and b1 aldehydes were formed.

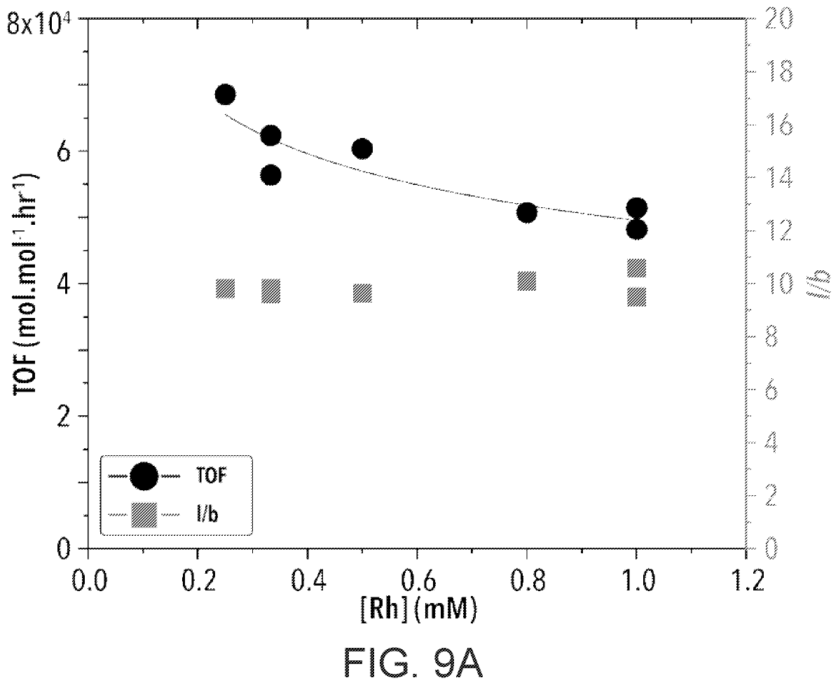

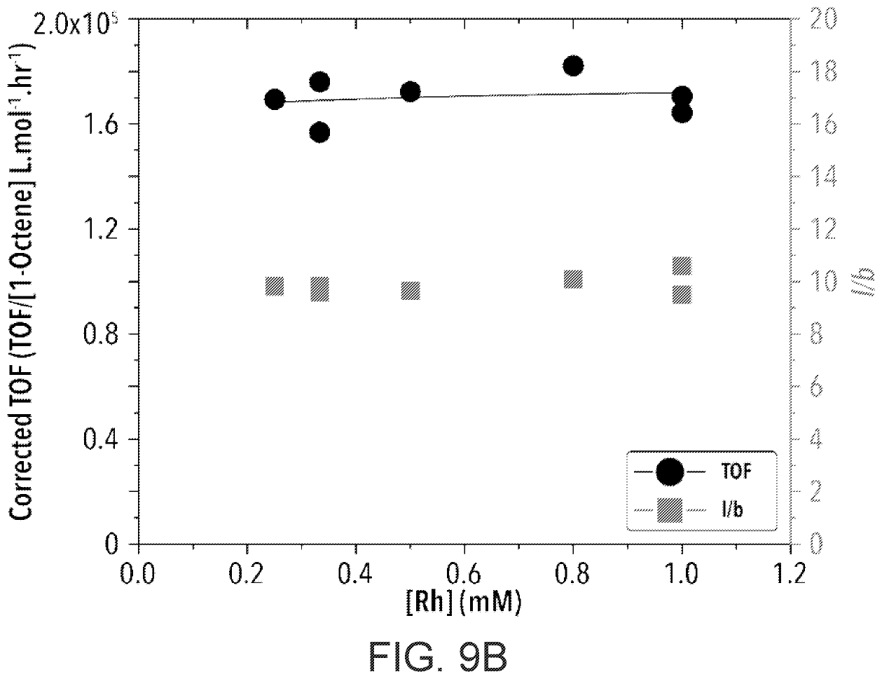

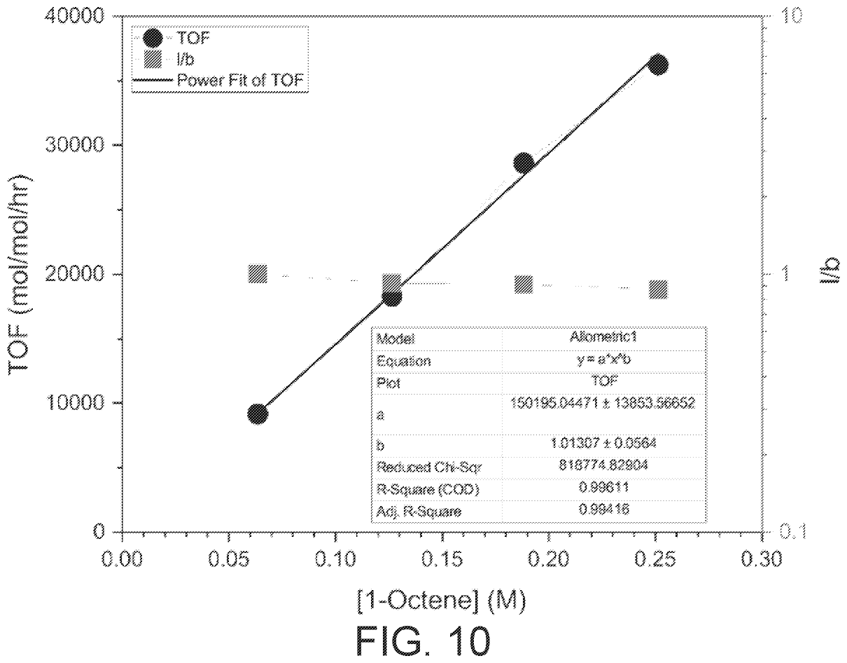

Next, the dependence of TOF and l/b on the catalyst loading at a constant [L], P_{CO}=7 psia, and T=75° C. was investigated using a pre-activated catalyst (FIG. 9). The TOF slightly decreased with increasing Rh loading (best fit power=0.8), indicating the possibility of the formation of the less active Rh dimers under this condition. The l/b remained unchanged at varying Rh loading. Based on these findings, it may be concluded that the l/b may be varied at a much larger range (0.75-13) by varying P_{CO} and [L], in addition to the reaction temperature.

### EXAMPLE 3

### Branched-Selective Hydroformylation in Flow

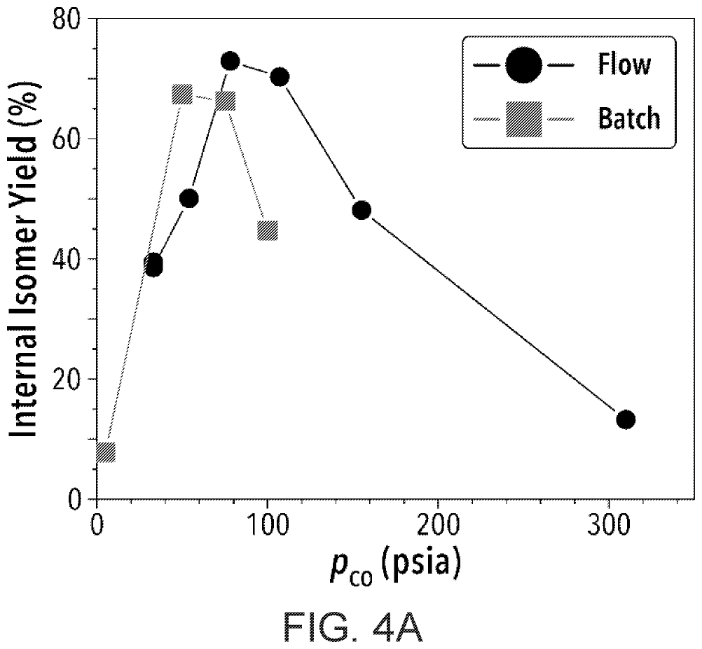

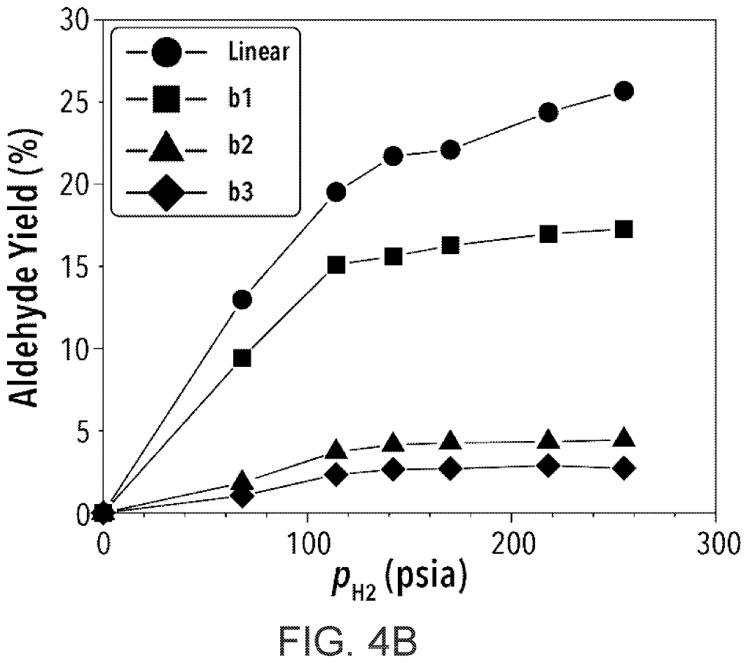

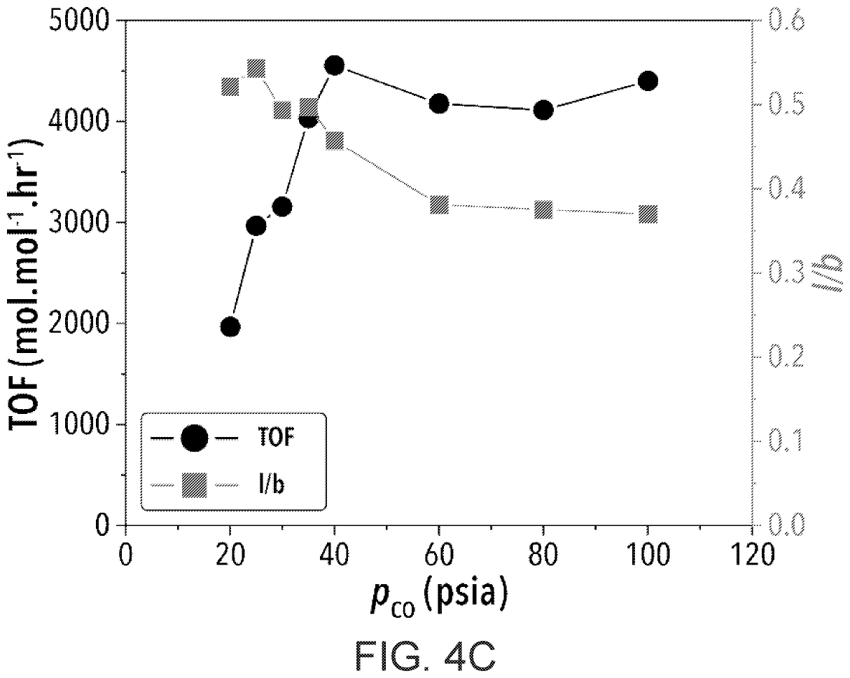

The results shown in FIGS. 2B and 3A indicate that both high P_{CO} and high reaction temperature affect the regioselectivity towards branched aldehydes with L. To understand the olefin isomerization activity under variable CO pressure, 1-octene was reacted with Rh/L at 110° C. without H₂ to halt the hydroformylation reaction. Rapid isomerization of the olefin occurred in the absence of H₂, and the isomerization activity went through a maximum between 50 psia and 150 psia P_{CO} in both flow and batch reactors, shown in FIG. 4A. The isomerization activity of Rh(CO)₂acac was also tested without L under P_{CO}=85 psia in the batch reactor, and the total aldehyde yield was less than 7% after 1 h at 110° C. This result supports the rate accelerating effect of L on Rh-catalyzed olefin isomerization under CO atmosphere.

Next, the effect of H₂ pressure, P_{H2}, on the yield of each produced aldehyde was studied at the maximum observed 1-octene isomerization P_{CO} (85 psia) in flow. Under these conditions, simultaneous hydroformylation of octene isomers occur, and the yield of all aldehydes increased in an apparent first order trend with P_{H2} up to 115 psia. At pressures higher than 115 psia, a slight increase in the yield of l was observed. However, an increase in yield was not observed for b2 and b3 (FIG. 4B). The l/b ratio for the variable P_{H2} experiments at P_{CO}=85 psia remained between 0.92 and 1.05 across the tested pressure range.

A varying P_{CO} experiment was also conducted for the hydroformylation of a cis and trans 2-octene mixture at 75° C. to investigate the effect of P_{CO} on the hydroformylation of the internal olefins with L (FIG. 4C). The TOF increased with P_{CO} up to 40 psia and did not show inhibition at higher P_{CO} values (FIG. 3A). The TOF was 10 times lower than that obtained in the hydroformylation of 1-octene at P_{CO}=40 psia (see FIG. 3A vs. FIG. 4C). The increase in P_{CO} also resulted in a decrease in l/b from 0.54 to 0.38 as P_{CO} was increased from 25 psia to 100 psia.

### EXAMPLE 4

### Modulating Hydroformylation Selectivity in a Flow Reactor

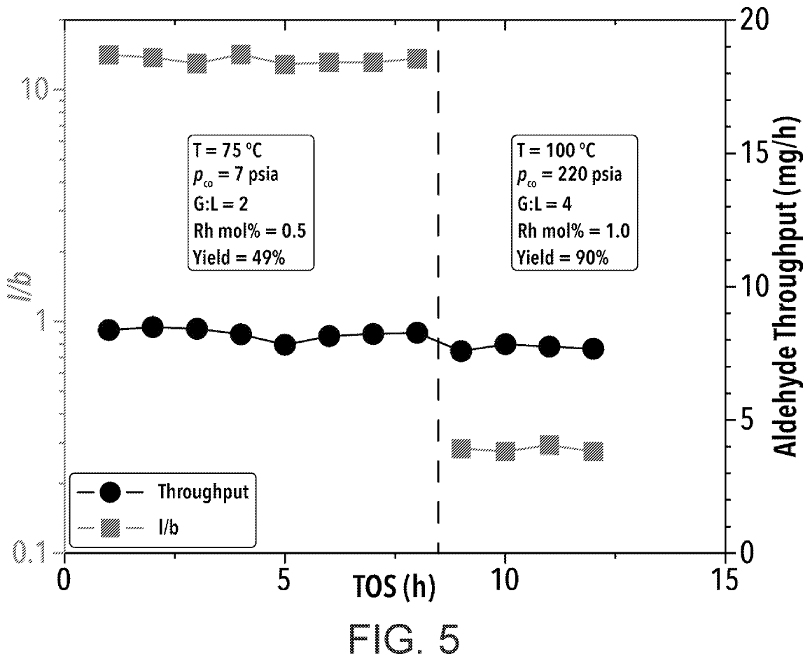

With the linear aldehyde-favoring, and branched aldehyde-favoring conditions in hand, on-line alternating aldehyde regioselectivity from a predominantly linear (l/b=13) to a predominantly branched (l/b=0.28) aldehyde was performed in the continuous flow reactor. For a total time on stream (TOS) of 12 h in 1-octene hydroformylation, the aldehyde throughput remained constant with a pre-activated Rh catalyst in the presence of L (FIG. 5). A l/b above 10 was obtained at a reaction temperature of 75° C. and a P_{CO} at 7 psia. The inlet gas to liquid volumetric ratio was set at 2 to allow for reasonable conversion, and to minimize the undesired olefin isomerization and hydrogenation in the pre-heat coil. In order to shift the aldehyde regioselectivity towards branched aldehydes (i.e., l/b=0.28), the reaction temperature of both the flow reactor and the preheat coil was raised to 100° C., and the P_{CO} was raised from 7 psia to 220 psia (TOS=8 h in FIG. 5). The inlet gas to liquid volumetric ratio was also increased from 2 to 4, and the Rh loading from 0.5 mol % to 1 mol % to maintain the same total aldehyde throughput. Total reaction pressure, 1-octene concentration, and ligand loading were maintained constant across the entire continuous flow hydroformylation operation (FIG. 5).

### EXAMPLE 5

### Modulating 1-Octene Hydroformylation Selectivity in a Batch Reactor

Following the alternating in-flow aldehyde regioselectivity enabled by L, the alternating regioselectivity capabilities of L was tested in a batch reactor with 1-octene and a pre-activated Rh/L catalyst at the L:Rh ratio of 40:1. For the linear-favoring experiments, based on the results shown in FIGS. 3A-3C, P_{CO} and reaction temperatures were set at 6 psia and 75° C., respectively. After a 2 h reaction, the total aldehyde yield was 50% (TOF 5000 mol ald·mol Rh⁻¹·h⁻¹), and l/b was 14.7 (93.6% linear) confirming the applicability of the rapid, linear aldehyde-favoring conditions in batch reactors (see Scheme 2, entry (1)).

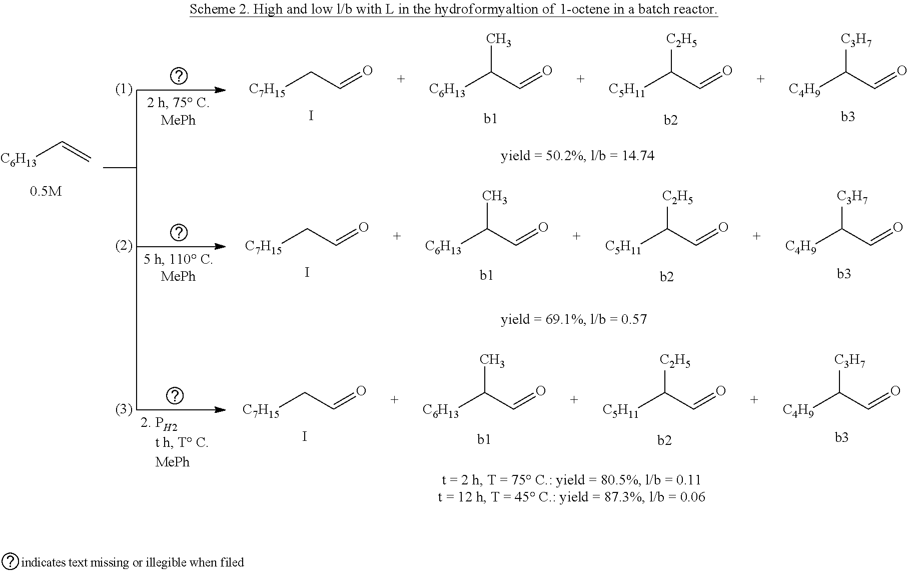

Next, the effect of ligand loading in the batch reactor with 0.05 mol % Rh was investigated. For these experiments, all catalysts, including the non-ligated control experiment, were pre-activated before use in the hydroformylation reaction. The total aldehyde yields ranged from 35% to 50% for all reactions, except for the reaction with the non-ligated Rh catalyst. For the reaction with a non-ligated Rh catalyst less than 5% aldehyde yield was afforded, further confirming the accelerating effect of L in the batch reactor (Table 2A).

TABLE 2A
Linear-selective hydroformylation of 1-octene at low CO pressure and temperature in the batch reactor.

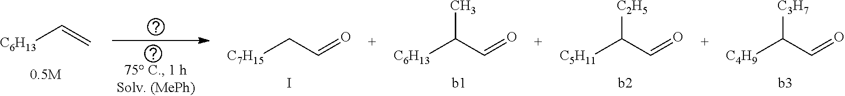

| | | | | | Hydroform. TOF | | |
|---|---|---|---|---|---|---|---|
| | | Aldehyde | | Unreacted 1- | mol ald · mol | Isom | Hydrogenation |
| | L:Rh | yield (%) | l/b | octene (%) | Rh⁻¹ · h⁻¹ | (%) | (%) |
| 1 | 0 | 4.8 | 2.64 | 7.3 | 94 | 74.3 | 13.5 |
| 2 | 4:1 | 44.9 | 1.92 | 0.3 | 866 | 31.0 | 23.9 |
| 3 | 10:1 | 37.3 | 4.88 | 0.6 | 721 | 35.1 | 26.9 |
| 4 | 20:1 | 36.3 | 9.72 | 0.7 | 700 | 37.0 | 26.1 |
| 5 | 40:1 | 49.2 | 14.74 | 0.6 | 950 | 29.6 | 20.6 |
| 6 | 100:1 | 39.1 | 15.05 | 0.5 | 754 | 36.3 | 24.1 |

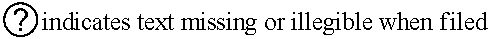

When [L] is less than 10 mM, a decrease in the l/b was observed, with a minimum of l/b=2 at [L]=1 mM. A relatively high n-octane formation and complete 1-octene conversion was observed for all experiments, except for the non-ligated catalyst reaction. To demonstrate the ability of ligand L to promote 1-octene hydroformylation at low l/b in the batch reactor, the reaction was performed at 110° C. to promote 1-octene isomerization to internal octenes that results in the formation of b1, b2, and b3. The hydrogen partial pressure (P_{H2}) was kept at 35 psia to retard the hydroformylation reaction to l relative to the isomerization. This approach resulted in an l/b ratio of 0.51 at 69% total aldehyde yield after 5 h (Scheme 2, entry (2)).

An alternative, two-step, one-pot reaction to obtain lower l/b was devised based on the observation that rapid olefin isomerization occurs under CO with ligand L (FIG. 4A). In this experiment, the substrate, catalyst, L, and solvent were heated to 110° C. under a CO atmosphere at 85 psia. The reaction was maintained at this temperature (110° C.) for 3 h. Next, the reaction was cooled down to 75° C. and H₂ was introduced into the batch reactor at 315 psia. After introduction of H₂ into the batch reactor, the reaction was continued for another 2 h. This method resulted in 80.5% aldehyde yield and l/b of 0.11. A further decrease in the l/b ratio to 0.06 was obtained when the second step temperature was cooled to 45° C., and when the reaction was permitted to proceed for 12 h with a total aldehyde yield of 87.3%. These reaction conditions allowed for the first time for a 94.3% branched aldehyde selectivity from 1-octene (Scheme 2, entry (3)).

Additional experiments further demonstrated that the hydroformylation of 1-octene could be switched from linear-selective to branched-selected (and vice versa) by manipulating temperature (T), carbon monoxide pressure (P_{CO}), and hydrogen pressure (P_{H2}) are manipulated, with all other variables remaining constant (Table 2B).

TABLE 2B
Alternating between linear-selective and branched-selective
hydroformylation of 1-octene by adjusting carbon monoxide pressure
(P_{CO}), hydrogen pressure (P_{H2}), and temperature (T). For
each experiment L:Rh = 40:1 and the catalyst was
preactivated. G/L = gas to liquid.

| | | | | | | | | alde- | |
|---|---|---|---|---|---|---|---|---|---|
| | | | | | | | | hyde | |
| | T | [1- | | Rh | | P_{CO} | P_{H2} | yield | |
| | (° C.) | octene] | | mol % | G/L | (psia) | (psia) | % | l/b |
| 1 | 75 | 0.2 | M | 0.1 | 8 | 8.50 | 341.50 | 32.08 | 2.50 |
| 2 | 75 | 0.2 | M | 0.1 | 6 | 8.50 | 341.50 | 30.36 | 5.00 |
| 3 | 75 | 0.2 | M | 0.1 | 4 | 8.50 | 341.50 | 28.26 | 6.05 |
| 4 | 75 | 0.2 | M | 0.1 | 2 | 8.50 | 341.50 | 19.81 | 7.71 |
| 5 | 75 | 0.05 | M | 0.5 | 4 | 8.5 | 341.5 | 28.28 | 2.71 |
| 6 | 75 | 0.05 | M | 0.5 | 6 | 8.5 | 341.5 | 33.78 | 3.30 |
| 7 | 75 | 0.05 | M | 0.5 | 2 | 8.5 | 341.5 | 39.69 | 10.53 |
| 8 | 75 | 0.05 | M | 0.5 | 2 | 8.5 | 341.5 | 42.59 | 8.48 |
| 9 | 110 | 0.05 | M | 0.5 | 2 | 315 | 35 | 37.27 | 0.61 |
| 10 | 110 | 0.05 | M | 0.5 | 2 | 298 | 52 | 50.51 | 0.59 |
| 11 | 110 | 0.05 | M | 0.5 | 2 | 298 | 52 | 52.66 | 0.59 |
| 12 | 110 | 0.05 | M | 0.5 | 2 | 323.7 | 26.3 | 51.37 | 0.54 |
| 13 | 110 | 0.05 | M | 0.5 | 2 | 280 | 70 | 58.62 | 0.51 |

| | | | | | | | | |
|---|---|---|---|---|---|---|---|---|
| 14 | 95 | 0.05 | 0.5 | 2.5 | 60 | 290 | 49.9 | 1.36 |
| 15 | 110 | 0.05 | 0.5 | 2.5 | 60 | 290 | 48.1 | 0.73 |
| 16 | 110 | 0.05 | 0.5 | 2.5 | 50 | 300 | 59.1 | 0.90 |

### EXAMPLE 6

### Comparison of Fluorophosphite Ligand (L) and Commonly Employed Mono and Bidentate Phosphine and Phosphite Ligands

In the next set of experiments, the flexibility of L was tested against representative mono and bidentate phosphine and phosphite ligands. The ligands tested are shown in Table 3.

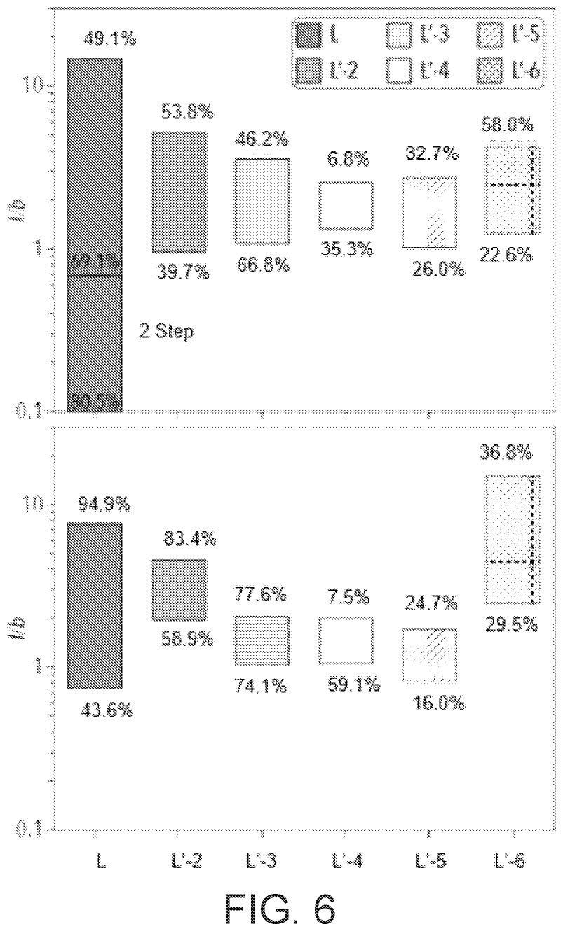

TABLE 3
Ligands tested for Example 6 (results shown in FIG. 6).

| Ligand | Structure | Name |
|---|---|---|
| L | 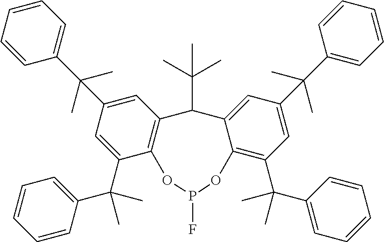 [SMILES: CC(C)(c1ccccc1)c1cc2c(c(C(C)(C)c3ccccc3)c1)OP(F)Oc1c(cc(C(C)(C)c3ccccc3)cc1C(C)(C)c1ccccc1)C2C(C)(C)C] | 12-(tert-butyl)-6-fluoro-2,4,8,10- tetrakis(2-phenylpropan-2-yl)-12H- dibenzo[d,g][1,3,2]dioxaphosphocine |
| L′-2 | 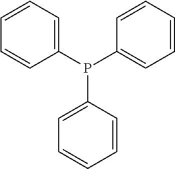 [SMILES: c1ccc(P(c2ccccc2)c2ccccc2)cc1] | triphenylphosphine |
| L′-3 | 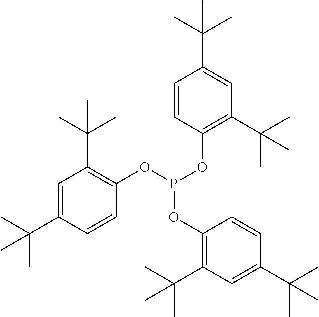 [SMILES: CC(C)(C)c1ccc(OP(Oc2ccc(C(C)(C)C)cc2C(C)(C)C)Oc2ccc(C(C)(C)C)cc2C(C)(C)C)c(C(C)(C)C)c1] | tris(2,4-di-t-butylphenyl)phosphite |
| L′-4 | 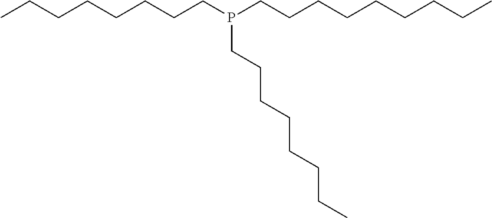 [SMILES: CCCCCCCCCP(CCCCCCCC)CCCCCCCC] | trioctylphosphine |
| L′-5 | 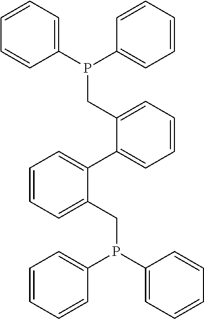 [SMILES: c1ccc(P(Cc2ccccc2-c2ccccc2CP(c2ccccc2)c2ccccc2)c2ccccc2)cc1] | 2,2′- bis((diphenylphosphaneyl)methyl)- 1,1′-biphenyl (BISBI) |
| L′-6 | 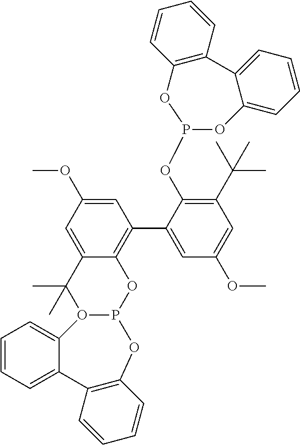 | 6,6′-[(3,3′-di-tert-butyl-5,5′- dimethoxy-[1,1′-biphenyl]-2,2′- diyl)bis(oxy)]bis(6H- dibenzo[d,f][1,3,2]dioxaphosphepine) (BiPhePhos) |

The ligands triphenylphosphine (L′-2), tris(2,4-di-t-butylphenyl)phosphite (L′-3), trioctylphosphine (L′-4), 2,2′-bis((diphenylphosphaneyl)methyl)-1,1′-biphenyl(BISBI) (L′-5), and 6,6′-[(3,3′-di-tert-butyl-5,5′-dimethoxy-[1,1′-biphenyl]-2,2′-diyl)bis(oxy)]bis(6H-dibenzo[d,f][1,3,2]dioxaphosphepine) (BiPhePhos) (L′-6) were tested under the identified linear- and branched-selective hydroformylation reaction conditions in FIG. 6 for the formylation of 1-octene (FIG. 6, top panel) and propylene (FIG. 6, bottom panel). The Rh loading was set at 0.05 mol % for each of the ligands. The obtained l/b flexibility range was limited to 0.96-5.27, 1.07-3.57, and 1.32-2.58 for L′-2, L′-3 and L′-4, respectively (FIG. 6).

For 1-octene, the total aldehyde yield was 53.8% and 46.2% for L′-2 and L′-3, respectively, under the linear aldehyde-favoring conditions, while L′-4 resulted in an only 6.8% total aldehyde yield under these conditions. For 1-octene, the difference in the performance of L when compared to L′-2, L′-3, and L′-4 under the branched aldehyde-favoring conditions may be attributed to the accelerating effect of L on the hydroformylation of the internal olefins as indicated by the higher content of aldehydes b2 and b3 with L (Table 4). Despite the high reaction temperature, the l/b remained high at 28.66 and 84.77 for the bidentate L′-5 and L′-6, respectively (Table 4).

TABLE 4
Bidentate L′-5 and L′-6 hydroformylation of 1-octene under branched-selective conditions.

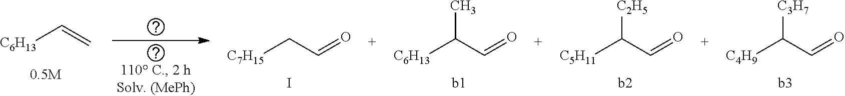

| Ligand | Ligand:Rh | Aldehyde yield (%) | l/b |
|---|---|---|---|
| L′-5 | 10:1 | 22.6 | 28.66 |
| L′-6 | 10:1 | 79.9 | 84.74 |

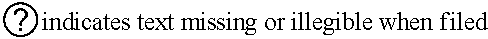

The Ligand:Rh ratio was lowered to 1.1:1 (2.2:1 phosphorus (P):Rh) for the L′-5 and L′-6 ligands. At this Ligand:Rh, the l/b spanned from 1.01-2.73 for L′-5 and from 1.23-4.29 for L′-6, indicating that bidentate ligands may not be made branched aldehyde-selective under the developed branched aldehyde-favoring condition.

### EXAMPLE 7

### Modulating Selectivity for Propylene Hydroformylation in a Batch Reactor

The selective formation of the commercially valuable isobutyraldehyde from propylene hydroformylation remains a challenge. The flexible hydroformylation of propylene in a batch reactor was explored with a pre-activated Rh catalyst with L. The branched-selective propylene hydroformylation was performed under a P_{CO} of 255 psia, a P_{H2} of 30 psia, and a reaction temperature of 120° C. These conditions resulted in a high selectivity towards isobutyraldehyde (l/b=0.74, 57.5% selectivity), as presented in Table 5A. The linear aldehyde-favoring hydroformylation of propylene to n-butyraldehyde was performed under a P_{CO} of 6 psia, a P_{H2} of 264 psia, and a temperature of 75° C. These conditions resulted in high selectivity towards n-butyraldehyde (l/b=5.01 and 6.60 at L:Rh=10:1 and 100:1, respectively). Increasing the propylene pressure, P_{C3H6}, to 60 psia resulted in a further increase in the aldehyde regioselectivity towards n-butyraldehyde (l/b=7.79). Alternating aldehyde regioselectivity comparisons of l/b for propylene hydroformylation using the cyclic monofluorophosphite ligand L versus other ligands shown in FIG. 6, showed that L enables access to a broader l/b range than the other ligands tested.

TABLE 5A
Alternating the aldehyde regioselectivity of the ligand L using propylene in a batch
reactor.

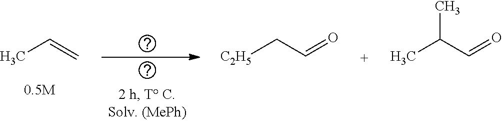

| | | | | | | | Hydroform. TOF |
|---|---|---|---|---|---|---|---|
| | | P_{C3H6} | P_{CO} | P_{H2} | | | mol ald · mol |
| | T(° C.) | (psia) | (psia) | (psia) | L:Rh | l/b | Rh⁻¹ · h⁻¹ |
| 1 | 120 | 15 | 255 | 30 | 10:1 | 0.74 | 511 |
| 2 | 75 | 30 | 6 | 264 | 10:1 | 5.01 | 214 |
| 3 | 75 | 30 | 6 | 264 | 100:1 | 6.60 | 236 |
| 4 | 75 | 60 | 6 | 234 | 100:1 | 7.79 | 361 |

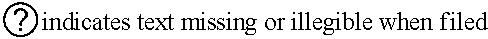

Additionally, experiments assessing the selectivity of propylenehydroformylation were conducted a constant CO pressure (P_{CO}), H₂ pressure (P_{H2}), and temperature (T) at different L:Rh values ranging from 0 to 100:1 for T=75° C. and T=110° C. The results are summarized in Tables 5B-5D.

TABLE 5B
Propylene hydroformylation with varying L:Rh at T = 75° C.,
P_{CO} = 6 psia, and P_{H2} = 264 psia. Catalyst complex preparation conditions:
0.25 mM Rh(CO)₂(acac) in toluene and fluorophosphite ligand L were preactivated at
85° C. for 1 hour under a pressure of 150 psia (1:1 P_{CO}:P_{H2}).

| | | | | | | | | |
|---|---|---|---|---|---|---|---|---|
| T (° C.) | 75 | 75 | 75 | 75 | 75 | 75 | 75 | 75 |
| P_{C}_{₃}_{H}_{₆} (psia) | 30 | 30 | 30 | 30 | 30 | 30 | 30 | 60 |
| P_{CO} (psia) | 6 | 6 | 6 | 6 | 6 | 6 | 6 | 6 |
| P_{H}_{₂} (psia) | 264 | 264 | 264 | 264 | 264 | 264 | 264 | 234 |
| Total pressure (psia) | 300 | 300 | 300 | 300 | 300 | 300 | 300 | 300 |
| L:Rh | 0 | 2.1:1 | 4:1 | 10:1 | 20:1 | 40:1 | 100:1 | 100:1 |
| time (h) | 2 | 2 | 2 | 2 | 2 | 2 | 2 | 2 |
| l/b (raw)¹ | 0.93 | 1.76 | 3.52 | 5.01 | 6.08 | 5.98 | 6.60 | 7.79 |
| Activity (h⁻¹) | 34.1 | 367.6 | 228.1 | 214.2 | 173.2 | 429.9 | 236.1 | 360.6 |

¹ratio of gas chromatography (GC) peak areas

TABLE 5C
Propylene hydroformylation with varying L:Rh at T = 110° C.,
P_{CO} = 6 psia, and P_{H2} = 264 psia. Catalyst complex preparation conditions:
0.25 mM Rh(CO)₂(acac) in toluene and fluorophosphite ligand L were preactivated at
85° C. for 1 hour under a pressure of 150 psia (1:1 P_{CO}:P_{H2}).

| | | | | | | | |
|---|---|---|---|---|---|---|---|
| T (° C.) | 110 | 110 | 110 | 110 | 110 | 110 | 110 |
| P_{C}_{₃}_{H}_{₆} (psia) | 30 | 30 | 30 | 30 | 30 | 30 | 30 |
| P_{CO} (psia) | 6 | 6 | 6 | 6 | 6 | 6 | 6 |
| P_{H}_{₂} (psia) | 264 | 264 | 264 | 264 | 264 | 264 | 264 |
| Total pressure (psia) | 300 | 300 | 300 | 300 | 300 | 300 | 300 |
| L:Rh | 0 | 2.1:1 | 4:1 | 10:1 | 20:1 | 40:1 | 100:1 |
| time (h) | 2 | 2 | 2 | 2 | 2 | 2 | 2 |
| l/b (raw)¹ | 0.82 | 1.30 | 2.23 | 3.38 | 4.52 | 5.19 | 5.94 |
| Activity (h⁻¹) | 51.3 | 231.7 | 296.4 | 285.2 | 342.8 | 281.4 | 255.8 |

¹ratio of gas chromatography (GC) peak areas

TABLE 5D
Propylene hydroformylation with varying L:Rh at T = 110° C., P_{CO} =
6 psia, and P_{H2} = 264 psia with a lower catalyst concentration (0.025
mM Rh instead of 0.25 mM Rh). Catalyst complex preparation conditions: 0.025
mM Rh(CO)₂(acac) in toluene and fluorophosphite ligand L were preactivated
at 85° C. for 1 hour under a pressure of 150 psia (1:1 P_{CO}:P_{H2}).

| | | | | | | | |
|---|---|---|---|---|---|---|---|
| T (° C.) | 110 | 110 | 110 | 110 | 110 | 110 | 110 |
| P_{C}_{₃}_{H}_{₆} (psia) | 30 | 30 | 30 | 30 | 30 | 30 | 30 |
| P_{CO} (psia) | 6 | 6 | 6 | 6 | 6 | 6 | 6 |
| P_{H}_{₂} (psia) | 264 | 264 | 264 | 264 | 264 | 264 | 264 |
| Total pressure (psia) | 300 | 300 | 300 | 300 | 300 | 300 | 300 |
| L:Rh | 0 | 2.1:1 | 4:1 | 10:1 | 20:1 | 40:1 | 100:1 |
| time (h) | 2 | 2 | 2 | 2 | 2 | 2 | 2 |
| l/b (raw)¹ | 0.85 | 0.94 | 1.11 | 1.35 | 1.79 | 2.39 | 3.12 |
| Activity (h⁻¹) | 273.4 | 955.1 | 2286.2 | 1592.2 | 1676.4 | 2264.1 | 2394.3 |

¹ratio of gas chromatography (GC) peak areas

### EXAMPLE 8

### Regioselective Hydroformylation of Internal Olefins

Due to the high activity of the pre-activated Rh/L catalyst for the hydroformylation of internal olefins, the efficacy of L in the regioselective hydroformylation of a cis-and trans-2-octene (70:30) mixture was investigated. To allow for a high rate of 2-octene hydroformylation to b1 and b2 relative to isomerization to 1-, 3-, and 4-octenes, the reaction temperature was set at 50° C., the P_{CO} was set at 75 psia, and the P_{H2} was set at 200 psia, where L:Rh=10:1. These conditions resulted in the regioselective hydroformylation of 2-octene to b1 and b2 without noticeable isomerization. The total aldehyde yield after 4 h was 61.5% (entry 1, Table 6). Extending the reaction time to 12 h increased the total aldehyde yield to 91.4% with a 93% selectivity of b1+b2 (entry 2, Table 6). The l/b ratio in this case was as low as 0.02 (i.e., 98% of the produced aldehydes were branched). The unique effect of ligand L on the regioselective hydroformylation of internal olefins is presented in entry 3, Table 6, where, in the absence of L, the l/b ratio increased to 0.11 at a total aldehyde yield of 28.3%. Increased olefin isomerization to the 3- and 4-octenes also occurred in the absence of L, which resulted in an increase in b3.

TABLE 6
Aldehyde regioselectivity tuning with a mixture of cis- and trans-2-octene.

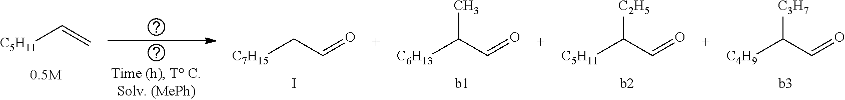

| | | | | | | | Hydroform. | |
|---|---|---|---|---|---|---|---|---|
| | | | | | | | TOF mol | Isomer |
| | T | P_{CO} | | Time | Aldehyde | l:b1:b2:b3 | ald · mol | yield |
| | (° C.) | (psia) | L:Rh | (h) | yield (%) | (l/b) | Rh⁻¹ · h⁻¹ | (%) |
| 1 | 50 | 75 | 10:1 | 4 | 61.5 | 1:23:15:1 | 297 | 38.3 |
| | | | | | | (0.03) | | |
| 2 | 50 | 75 | 10:1 | 12 | 91.4 | 1:29:22:3 | 147 | 2.53 |
| | | | | | | (0.02) | | |
| 3 | 50 | 75 | 0 | 12 | 28.3 | 3:21:6:1 | 46 | 20.40 |
| | | | | | | (0.11) | | |
| 4 | 100 | 6 | 40:1 | 1 | 39.1 | 18:8:2:1 | 377 | 11.85 |
| | | | | | | (1.62) | | |

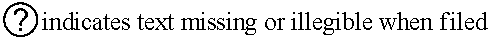

Ligand L may become linear-selective for the hydroformylation of 2-octene by altering the temperature, P_{CO}, P_{H2}, and L:Rh ratio (100° C., P_{CO}=6 psia, P_{H2}=200 psia, and L:Rh=40:1). Hydroformylation at the aforementioned reaction conditions resulted in a l/b of 1.62 (1.62=62% selectivity to l) at 39.1% total aldehyde yield (Table 6, entry 4). The results in Table 6, entries 1 and 3, illustrate the flexibility of ligand L in tuning of regioselective aldehyde formation from internal olefins.

### EXAMPLE 9

### In-Flow Hydroformylation vs. Batch Hydroformylation

Traditionally, hydroformylation reactions are studied in pressurized autoclaves that have relatively large volumes in the order of 10-100 mL for bench scale units. Autoclaves are often pressurized before heating to minimize evaporation, and the time needed to reach the target temperature is relatively long (˜10 min). This limitation hampers the ability of batch reactors to elucidate the effect of reaction temperature on the catalyst/ligand activity and aldehyde selectivity. Furthermore, accurate pressure control in autoclaves may be challenging due to the gas thermal expansion upon heating, and pressure changes as the reaction consumes or produces gaseous species.

The developed flow chemistry platform described herein is equipped with a continuous gas-liquid segmented flow reactor that allows for rapid reaction parameter space mapping for the catalyst activity and aldehyde regioselectivity. The utilized flow reactor allows for gas-liquid segmentation at the target reaction conditions with high surface area to volume ratios, thereby accelerating both heat and mass transfer rates. These intensified heat and mass transport rates, in combination with the precise control over reaction conditions, allow for accurate investigation of reactivity and selectivity at early reaction times (5-10 min), which is challenging in batch reactors. Such early-time monitoring and analysis of the hydroformylation reactions in the flow reactor may facilitate understanding of the change in reactivity attributed to the catalyst activation. These advantages make the developed flow chemistry platform a suitable tool for accurate mechanistic investigations of high pressure/temperature gas-liquid reactions, particularly hydroformylation of olefins.

Cyclic fluorophosphites are strong x-acceptor ligands and 8-membered cycles exhibit reasonable stability. However, their performance as a hydroformylation ligand is not well explored. The hydroformylation mechanism and the active catalyst species depend heavily on the ligand structure and the reaction conditions. A higher catalytic activity was observed in the flow reactor relative to the batch reactor (FIG. 2A).

### EXAMPLE 10

### Effect of Reaction Temperature on the Catalyst Reactivity and Aldehyde Regioselectivity

With a H₂:CO ratio of 1, the maximum total aldehyde yield was achieved at 95° C. (FIG. 2A). At higher temperatures, rapid isomerization of 1-octene occurred relative to hydroformylation (FIG. 2B). While internal octenes may undergo hydroformylation in the presence of L, their rate of hydroformylation is 10 times slower than that of 1-octene (FIGS. 3A and 4C). This shift in the double bond position lowers the total aldehyde yields as temperature increases above 95° C. Olefin isomerization may also be attributed to the change in the slope of the yield vs. time curves at t_{R}>30 min in FIG. 2A.

The catalyst pre-activation with L appears to be more sensitive to temperature than the hydroformylation reaction. At 75° C., the effect of catalyst pre-activation may be observed up to t_{R}=30 min. However, it the effect of catalyst pre-activation is not noticeable at 110° C. even at residence times as low as 5 min (FIG. 2A). It should be noted here that the catalyst pre-activation accelerates the olefin isomerization as well which results in a slight decrease in the l/b ratio at t_{R}>30 min (i.e., l/b=0.96 vs. 1.35 without the pre-activation).

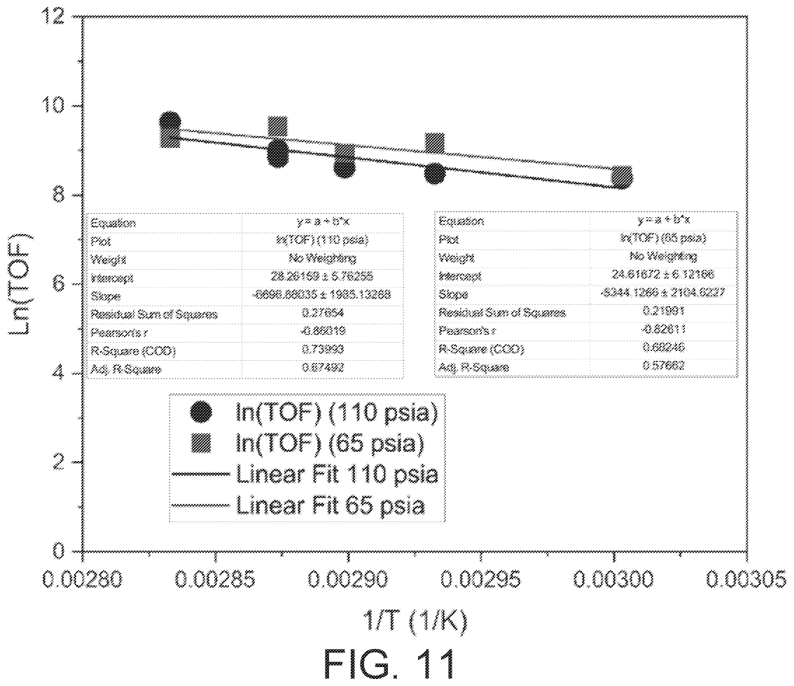

Aside from olefin isomerization, higher reaction temperature appears to favor the branched aldehyde formation from terminal olefin in the presence of L by destabilizing the bis-ligated L-Rh-L species and convert them to the mono-ligated L-Rh carbonyl species. This effect is likely the reason for the decrease in the l/b and TOF observed in FIG. 3C with 1-octene and the decrease in l/b with propylene in Table 6. Indeed, when reaction temperature screening experiments was performed at higher P_{CO}, (65 psia and 107 psia), the TOF increased with the reaction temperature and activation barriers of 44.4 kJ·mol⁻¹ and 55.7 kJ·mol⁻¹ were measured, respectively (FIG. 11). This result suggests that the measured activation barrier increases with increasing the CO pressure when the hydroformylation conditions favor the formation of the branched aldehyde.

### EXAMPLE 11

### Effect of Syngas Composition on the Catalyst Activity and Aldehyde Regioselectivity

An increase in the l/b ratio in the presence of L occurs in the low P_{CO} range (7 psia to 60 psia) where an l/b ratio as high as 14 may be obtained in both flow and batch reactors (FIG. 5 and Scheme 2). This shift in the aldehyde regioselectivity may be attributed to the shift in the L ligation mechanism from the mono-ligated species (L-Rh-CO) to the bis-ligated (L₂-Rh) species. Due to the high steric hindrance provided by L around the Rh atom, the bis-ligated species may favor the formation of l over b1. However, the steric hinderance is also responsible for the destabilization of these species at high CO pressures (>60 psia). The unique ability of L to alternate between the two ligation modes gives it a wide l/b span, especially compared to other ligands (FIG. 6). At CO pressures below 12 psia, increasing the ligand concentration impacts both the aldehyde regioselectivity and the catalyst activity (FIG. 3B), indicating that the equilibrium between the two catalyst ligation modes with L is dependent on the CO pressure, as well as the ligand concentration in the solution or the [L]/[CO] ratio, and less dependent on the L:Rh ratio.

Carbon monoxide inhibits the formation of the linear aldehyde at P_{CO}>12 psia. Similar inhibition was observed in the olefin isomerization experiment with no H₂ at P_{CO}>100 psia (FIG. 4A). The inhibition of both terminal olefin hydroformylation and isomerization by CO may be attributed to the competitive coordination of CO to Rh with either the olefin or the ligand or both (see mechanism section below). At CO pressures below 12 psia in the flow reactor, the hydroformylation becomes limited by CO availability in the liquid phase as indicated by the decrease in the aldehyde formation at P_{CO}=7 psia (FIG. 3A).

The rate of formation of all aldehydes appeared to be first order with respect to H₂ up to P_{H2}=115 psia where P_{CO} remains constant at 85 psia. The reaction rate dependence on P_{H2} suggests that the reaction is limited by either the oxidative addition of molecular H₂ to the Rh acyl or the reductive elimination of the aldehyde. At higher P_{H2}, the formation of l and b1 appears to increase, while the formation of b2 and b3 appears to remain constant. As olefin isomerization occurs at P_{H2}=0, higher P_{H2} promotes the hydroformylation of the olefin over olefin isomerization, thus increasing l/b ratios at a constant total aldehyde yield (FIG. 5). As illustrated in Scheme 2, the effects of hydrogen pressure and reaction temperature demonstrated in the experiments conducted in the flow reactor are directly applicable to batch reactions as demonstrated by (2) the l/b ratio of 0.57 in one-step batch reaction at high reaction temperature (110° C.) and low P_{H2} (35 psia), and (3) the low l/b ratio of 0.06 with a two-step, single batch process with an inter-stage cooling (Scheme 2, entries (2) and (3)).

Furthermore, high P_{CO} (75 psia) was observed to promote the selective hydroformylation of 2-octene to b1 and b2 with minimum formation of the l and b3 isomers. The reaction temperature was maintained at 50° C. to minimize olefin isomerization. However, this P_{CO} was efficient in preventing the formation of linear aldehydes even at L:Rh ratios as high as 10:1 (Table 6).

### EXAMPLE 12

### Investigation of Ligands L2-L5

Ligands L2-L5 and their corresponding NMR assignments are shown in Table 7. The ligands were prepared using the methods described in the general methods section above with the appropriate starting materials. After the synthesis and characterization of ligands L2-L5, l/b modulation conditions were investigated, and the results are shown below in Tables 8-11.

TABLE 7
Exemplary ligands L2-L5.

| Ligand | Structure | NMR Characterization |
|---|---|---|
| L2 | 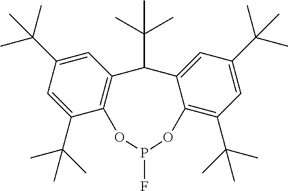 [SMILES: CC(C)(C)c1cc2c(c(C(C)(C)C)c1)OP(F)Oc1c(cc(C(C)(C)C)cc1C(C)(C)C)C2C(C)(C)C] | ¹H NMR (600 MHz, C₆D₆-d₆ Benzene): δ 7.83 (d, 2H, ArH), δ 7.38 (d, 2H, ArH), δ 4.99 (s, 1H, bridge C—H), δ 1.46-1.19 (m, 45H, 5 × C(CH₃)₃). ³¹P NMR (243 MHz): δ 136.43 (d, J = 1261.2 Hz), 105.78 (d, J = 1241.7 Hz). ¹⁹F NMR (564 MHz): δ −62.71 (d, J = 1240.8 Hz), −71.53 (d, J = 1257.7 Hz). |
| L3 | 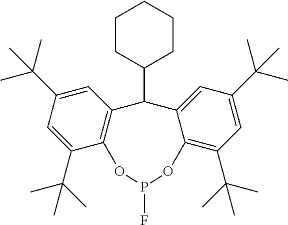 [SMILES: CC(C)(C)c1cc2c(c(C(C)(C)C)c1)OP(F)Oc1c(cc(C(C)(C)C)cc1C(C)(C)C)C2C1CCCCC1] | ¹H NMR (600 MHz, C₇D₈-d₈ Toluene): δ 7.53 (d, 2H, ArH), δ 7.37 (d, 2H, ArH), δ 4.58 (d, 1H, bridge C—H), δ 1.52-1.32 (m, 36H, 4 × C(CH₃)₃), δ 2.16-1.62 (m, 11H, Cyclohexyl 5 × CH₂, 1 × C—H). ³¹P NMR (243 MHz) δ 104.84 (d, J = 1232.0 Hz). ¹⁹F NMR (564 MHz): δ −63.90 (d, J = 1235.2 Hz), −69.91 (d, J = 1246.4 Hz), −71.58 (d, J = 1257.7 Hz). |
| L4 | 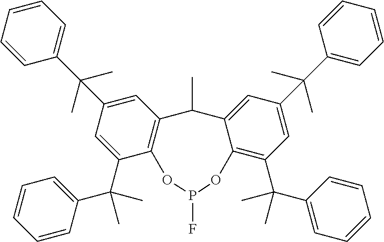 [SMILES: CC1c2cc(C(C)(C)c3ccccc3)cc(C(C)(C)c3ccccc3)c2OP(F)Oc2c1cc(C(C)(C)c1ccccc1)cc2C(C)(C)c1ccccc1] | ¹H NMR (600 MHz, C₇D₈-d₈ Toluene): δ 7.36- 7.01 (m, 24H, ArH), δ 4.42 (q, 1H, bridge C—H), δ 1.64 (s, 24H, 4 × C(CH₃)₂), δ 1.54 (d, 3H, bridge CH₃). |
| L5 | 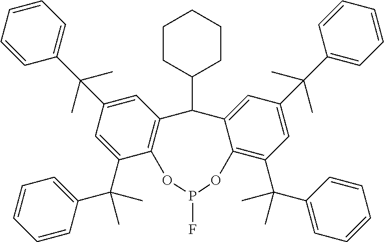 [SMILES: CC(C)(c1ccccc1)c1cc2c(c(C(C)(C)c3ccccc3)c1)OP(F)Oc1c(cc(C(C)(C)c3ccccc3)cc1C(C)(C)c1ccccc1)C2C1CCCCC1] | ¹H NMR (600 MHz, C₇D₈-d₈ Toluene): δ 7.24 7.01 (m, 24H, ArH), δ 4.14 (d, 1H, bridge C—H), δ 2.15 (m, 1H, Cyclohexyl C—H), δ 1.71-1.55 (m, 34H, Cyclohexyl5 × CH₂, 4 × C(CH₃)₂). ³¹P NMR (243 MHz): δ 101.48 (d, J = 1268.5 Hz). ¹⁹F NMR (564 MHz): δ −66.88 (d, J = 1263.4 Hz), −67.27 (d, J = 1302.8 Hz), −68.35 (d, J = 1308.5 Hz). |

TABLE 8
Results of L2 l/b studies for 1-octene hydroformylation.

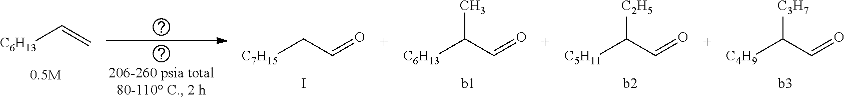

| | | | | | | | % | % | TOF (mol |
|---|---|---|---|---|---|---|---|---|---|
| | | | | | | | aldehyde | unreacted | aldehyde · mol |
| | L:Rh | T (° C.) | P_{CO} | P_{H2} | l/b* | l/b** | conversion | 1-octene | Rh⁻¹ · h⁻¹) |
| 1 | 10:1 | 80 | 65 | 195 | 2.24 | 2.28 | 86.0 | 0.9 | 860 |
| 2 | 10:1 | 110 | 130 | 130 | 1.34 | 1.67 | 87.2 | 3.0 | 872 |
| 3 | 10:1 | 80 | 130 | 130 | 1.68 | 1.87 | 83.2 | 3.1 | 832 |
| 4 | 10:1 | 95 | 130 | 130 | 1.08 | 1.49 | 87.2 | 2.6 | 872 |
| 5 | 20:1 | 80 | 130 | 130 | 2.07 | 2.20 | 84.4 | 1.0 | 844 |
| 6 | 10:1 | 110 | 200 | 35 | 0.85 | 1.24 | 87.2 | 2.6 | 349 |
| 7 | 400:1 | 80 | 6 | 200 | 30.31 | 30.31 | 23.7 | 36.0 | 2367 |

*l/b = peak area of nonanal (l) to all branched aldehydes (b1 + b2 + b3).
**l/b = peak area of nonanal (l) to 2-methyloctanal (b1)

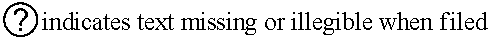

TABLE 9
Results of L3 l/b studies for 1-octene hydroformylation.

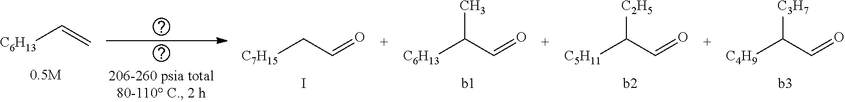

| | | | | | | | | | TOF (mol |
|---|---|---|---|---|---|---|---|---|---|
| | | T | | | | | % aldehyde | % unreacted | aldehyde · mol |
| | L:Rh | (° C.) | P_{CO} | P_{H2} | l/b* | l/b** | conversion | 1-octene | Rh⁻¹ · h⁻¹) |
| 1 | 10:1 | 80 | 65 | 195 | 2.31 | 2.57 | 81.5 | 3.7 | 815 |
| 2 | 10:1 | 80 | 130 | 130 | 1.65 | 1.79 | 82.5 | 5.0 | 825 |
| 3 | 10:1 | 95 | 130 | 130 | 1.15 | 1.51 | 82.9 | 4.6 | 829 |
| 4 | 10:1 | 110 | 130 | 130 | 0.86 | 1.28 | 89.0 | 3.0 | 890 |
| 5 | 10:1 | 110 | 200 | 35 | 1.16 | 1.54 | 84.2 | 3.5 | 337 |
| 6 | 400:1 | 80 | 6 | 200 | 8.41 | 8.41 | 5.7 | 89.8 | 571 |

*l/b = peak area of nonanal (l) to all branched aldehydes (b1 + b2 + b3).
**l/b = peak area of nonanal (l) to 2-methyloctanal (b1)

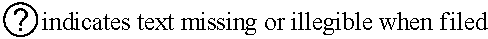

TABLE 10
Results of L4 l/b studies for 1-octene hydroformylation.

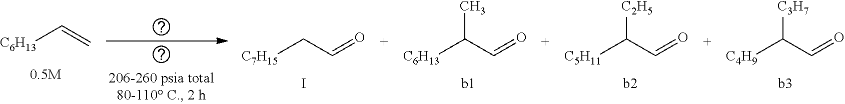

| | | | | | | | % | % | TOF (mol |
|---|---|---|---|---|---|---|---|---|---|
| | | | | | | | aldehyde | unreacted | aldehyde · mol |
| | L:Rh | T (° C.) | P_{CO} | P_{H2} | l/b* | l/b** | conversion | 1-octene | Rh⁻¹ · h⁻¹) |
| 1 | 10:1 | 80 | 65 | 195 | 2.16 | 2.69 | 82.3 | 4.6 | 823 |
| 2 | 10:1 | 80 | 130 | 130 | 2.25 | 2.25 | 32.7 | 64.2 | 327 |
| 3 | 10:1 | 95 | 130 | 130 | 1.22 | 1.63 | 78.4 | 3.9 | 784 |
| 4 | 10:1 | 110 | 130 | 130 | 0.99 | 1.45 | 88.2 | 1.5 | 882 |
| 5 | 10:1 | 110 | 200 | 35 | 1.41 | 1.79 | 70.9 | 4.3 | 283 |
| 6 | 400:1 | 80 | 6 | 200 | 27.27 | 27.27 | 28.4 | 56.7 | 2842 |

*l/b = peak area of nonanal (l) to all branched aldehydes (b1 + b2 + b3).
**l/b = peak area of nonanal (l) to 2-methyloctanal (b1)

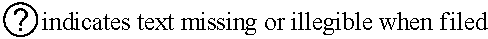

TABLE 11
Results of L5 l/b studies for 1-octene hydroformylation.

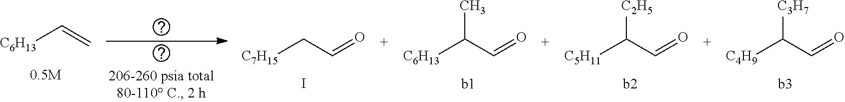

| | | | | | | | | | TOF (mol |
|---|---|---|---|---|---|---|---|---|---|
| | | | | | | | % aldehyde | % unreacted | aldehyde · mol |
| | L:Rh | T (º C.) | P_{CO} | P_{H2} | l/b* | l/b** | conversion | 1-octene | Rh⁻¹ · h⁻¹) |
| 1 | 10:1 | 80 | 130 | 130 | 1.35 | 1.56 | 75.2 | 5.9 | 752 |
| 2 | 10:1 | 95 | 130 | 130 | 0.83 | 1.20 | 83.2 | 4.8 | 832 |
| 3 | 10:1 | 110 | 130 | 130 | 0.67 | 1.05 | 88.2 | 4.0 | 882 |
| 4 | 10:1 | 80 | 65 | 195 | 1.14 | 1.53 | 83.7 | 5.2 | 837 |
| 5 | 10:1 | 110 | 200 | 35 | 0.95 | 1.29 | 69.4 | 4.6 | 278 |
| 6 | 400:1 | 80 | 6 | 200 | 9.04 | 9.04 | 34.1 | 22.4 | 3406 |

*l/b = peak area of nonanal (l) to all branched aldehydes (b1 + b2 + b3).
**l/b = peak area of nonanal (l) to 2-methyloctanal (b1)

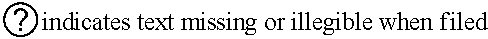

The foregoing description of the specific aspects will so fully reveal the general nature of the technology that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the hydroformylation process are set out in the following numbered embodiments. The first embodiment is denoted E1, the second embodiment is denoted E2 and so forth.

E1. A process for preparing linear and branched aldehydes, the process comprising:
contacting an olefin with hydrogen (H₂) and carbon monoxide (CO) in the presence of a catalyst solution, the catalyst solution comprising:
   a hydroformylation solvent;
   a rhodium (Rh) source; and
   a fluorophosphite ligand of formula (I):

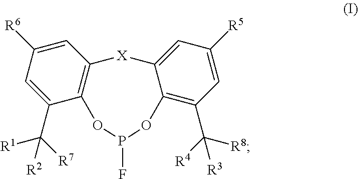

wherein:
X is —CH(R)—;
R is independently C₁₋₆alkyl, a 3- to 7-membered carbocycle, or a phenyl, where the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl;
R¹, R², R³, R⁴ are each independently C₁₋₆alkyl, C₁₋₂haloalkyl, hydrogen, halogen, or —CN;
R⁵ and R⁶ are each

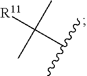

R¹¹ is a C₁₋₆alkyl, a 3- to 6-membered carbocycle, or a phenyl, wherein the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl;
R⁷ and R⁸ are each independently C₁₋₆alkyl, a 6- to 12-membered aryl, or a 3- to 6-membered carbocycle, wherein the 6- to 12-membered aryl and 3- to 6-membered carbocycle are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl; and
   wherein the contacting of the olefin with the H₂ and the CO in the presence of the catalyst solution at:
a temperature ranging from 95° C. to 130° C.,
a carbon monoxide partial pressure (P_{CO}) ranging from 110 psia to 350 psia, and
a hydrogen partial pressure (P_{H2}) ranging from 20 psia to 150 psia, produces a ratio of linear aldehydes to branched aldehydes (l/b) of less than 1.0; and
   wherein the contacting of the olefin with the H₂ and the CO in the presence of the catalyst solution at:
a temperature ranging from 60° C. to 100° C.,
a P_{CO} ranging from 5 psia to 150 psia, and
a P_{H2} ranging from 100 psia to 350 psia,
   produces l/b of greater than 1.0.
   E2. The process of E1, wherein the contacting of the olefin with the H₂ and the CO in the presence of the catalyst solution at:
a temperature ranging from 100° C. to 120° C.,
a P_{CO} ranging from 130 psia to 230 psia, and
a P_{H2} ranging from 30 psia to 130 psia,
   produces a l/b of less than 0.90.
   E3. The process of E1 or E2, wherein the contacting of the olefin with the H₂ and the CO in the presence of the catalyst solution at:
a temperature ranging from 100° C. to 120° C.,
a P_{CO} ranging from 200 psia to 300 psia, and
a P_{H2} ranging from 20 psia to 50 psia,
   produces a l/b of less than 0.60.
   E4. The process any one of E1-E3, wherein the contacting of the olefin with the H₂ and the CO in the presence of the catalyst solution at:
a temperature ranging from 75° C. to 85° C.,
a P_{CO} ranging from 5 psia to 10 psia, and
a P_{H2} ranging from 200 to 300 psia,
   produces a l/b of greater than 1.6.
   E5. The process of any one of E1-E4, wherein the contacting of the olefin with the H₂ and the CO in the presence of the catalyst solution at:
a temperature ranging from 75° C. to 85° C.,
a P_{CO} ranging from 50 psia to 150 psia, and
a P_{H2} ranging from 110 psia to 210 psia,
   produces a l/b of greater than 1.6.
   E6. The process of any one of E1-E5, wherein the olefin is a C₃₋₂₀alkene or a C₃₋₈cycloalkene.
   E7. The process of any one of E1-E6, wherein the olefin is a C₃₋₁₀alkene.
   E8. The process of any one of E1-E7, wherein the olefin is propylene, 1-octene, or 2-octene.
   E9. The process of any one of E1-E8, wherein the ratio of Rh to the ligand of formula (I) ranges from 10:1 to 400:1.
   E10. The process of any one of E1-E9, wherein the ratio of Rh to the ligand of formula (I) ranges from 10:1 to 100:1.
   E11. The process of any one of E1-E10, wherein the ratio of Rh to the ligand of formula (I) ranges from 10:1 to 50:1.
   E12. The process of any one of E1-E11, wherein the hydroformylation solvent is toluene.
   E13. The process of any one of E1-E12, wherein R is C₁₋₄alkyl or a 3- to 6-membered carbocycle.
   E14. The process of any one of E1-E13, wherein X is

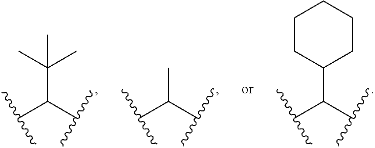

E15. The process of any one of E1-E14, wherein R¹, R², R³, and R⁴ are each C₁₋₄alkyl.
E16. The process of any one of E1-E15, wherein R¹, R², R³, and R⁴ are each —CH₃.
E17. The process of any one of E1-E16, wherein R5 and R6 are each independently

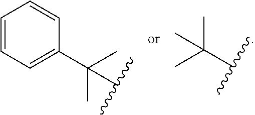

E18. The process of any one of E1-E17, wherein R⁷ and R⁸ are each C₁₋₄alkyl or the unsubstituted or substituted phenyl.
E19. The process of any one of E1-E18, wherein R⁷ and R⁸ are each —CH₃ or unsubstituted phenyl.
E20. The process of any one of E1-E19, wherein the fluorophosphite ligand of formula (I) is a ligand of formula (Ia):

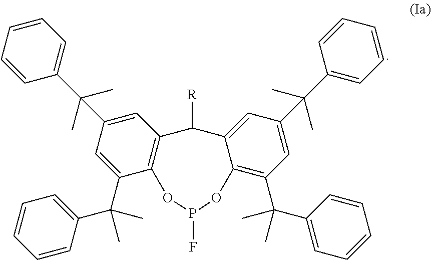

E21. The process of any one of E1-E20, wherein the fluorophosphite ligand of formula (I) is a ligand of formula (Ib):

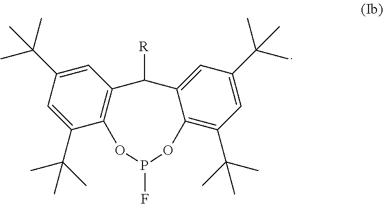

E22. The process of any one of E1-E21, wherein the fluorophosphite ligand of formula (I) is

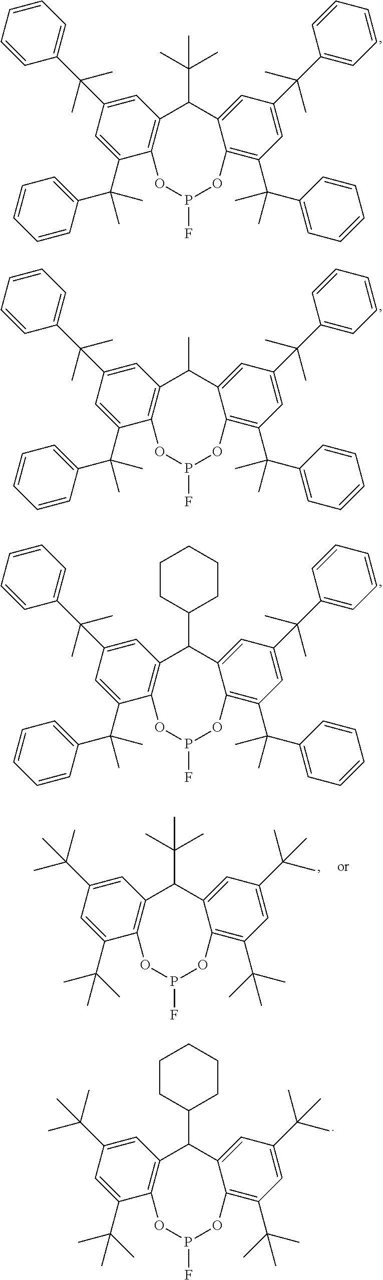

1. A process for preparing linear and branched aldehydes, the process comprising:
contacting an olefin with hydrogen (H₂) and carbon monoxide (CO) in the presence of a catalyst solution, the catalyst solution comprising:
a hydroformylation solvent;
a rhodium (Rh) source; and
a fluorophosphite ligand of formula (I): 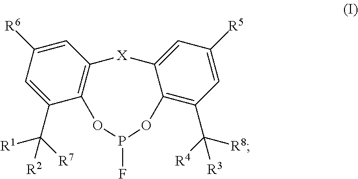
wherein:
X is —CH(R)—;
R is independently C₁₋₆alkyl, a 3- to 7-membered carbocycle, or a phenyl, where the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and-OC₁₋₂haloalkyl;
R¹, R², R³, R⁴ are each independently C₁₋₆alkyl, C₁₋₂haloalkyl, hydrogen, halogen, or —CN;
R⁵ and R⁶ are each 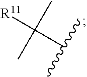
R¹¹ is a C₁₋₆alkyl, a 3- to 6-membered carbocycle, or a phenyl, wherein the 3- to 6-membered carbocycle or phenyl are each unsubstituted or substituted with 1-4 substituents each independently selected from the group consisting of halogen, —CN, C₁₋₄alkyl, C₁₋₂haloalkyl, —OC₁₋₄alkyl, and —OC₁₋₂haloalkyl;
R⁷ and R⁸ are each independently C₁₋₆alkyl, a 6- to 12-membered aryl, or a 3- to 6-membered carbocycle, wherein the 6- to 12-membered aryl and 3- to 6-membered carbocycle are each unsubstituted or substituted with 1-4 substituents each independently selected from the group consisting of halogen, —CN, C₁₋₄alkyl,
C₁₋₂haloalkyl, —OC₁₋₄alkyl, and —OC₁₋₂haloalkyl; and
wherein the contacting of the olefin with the H₂ and the CO in the presence of the catalyst solution at:
a temperature ranging from 95° C. to 130° C.,
a carbon monoxide partial pressure (P_{CO}) ranging from 110 psia to 350 psia, and
a hydrogen partial pressure (P_{H2}) ranging from 20 psia to 150 psia,
produces a ratio of linear aldehydes to branched aldehydes (l/b) of less than 1.0; and
wherein the contacting of the olefin with the H₂ and the CO in the presence of the catalyst solution at:
a temperature ranging from 60° C. to 100° C.,
a P_{CO} ranging from 5 psia to 150 psia, and
a P_{H2} ranging from 100 psia to 350 psia,
produces a l/b of greater than 1.0.

2. The process of claim 1, wherein the contacting of the olefin with the H₂ and the CO in the presence of the catalyst solution at:
a temperature ranging from 100° C. to 120° C.,
a P_{CO} ranging from 130 psia to 230 psia, and
a P_{H2} ranging from 30 psia to 130 psia,
produces a l/b of less than 0.90.

3. The process of claim 1, wherein the contacting of the olefin with the H₂ and the CO in the presence of the catalyst solution at:
a temperature ranging from 100° C. to 120° C.,
a P_{CO} ranging from 200 psia to 300 psia, and
a P_{H2} ranging from 20 psia to 50 psia,
produces a l/b of less than 0.60.

4. The process of claim 1, wherein the contacting of the olefin with the H₂ and the CO in the presence of the catalyst solution at:
a temperature ranging from 75° C. to 85° C.,
a P_{CO} ranging from 5 psia to 10 psia, and
a P_{H2} ranging from 200 to 300 psia,
produces a l/b of greater than 1.6.

5. The process of claim 1, wherein the contacting of the olefin with the H₂ and the CO in the presence of the catalyst solution at:
a temperature ranging from 75° C. to 85° C.,
a P_{CO} ranging from 50 to 150 psia, and
a P_{H2} ranging from 110 to 210 psia,
produces a l/b of greater than 1.6.

6. The process of claim 1, wherein the olefin is a C₃₋₂₀alkene or a C₃₋₈cycloalkene.

7. (canceled)

8. The process of claim 6, wherein the olefin is propylene, 1-octene, or 2-octene.

9. The process of claim 1, wherein the ratio of Rh to the ligand of formula (I) ranges from 10:1 to 400:1.

10. (canceled)

11. The process of claim 9, wherein the ratio of Rh to the ligand of formula (I) ranges from 10:1 to 50:1.

12. The process of claim 1, wherein the hydroformylation solvent is toluene.

13. The process of claim 1, wherein R is C₁₋₄alkyl or a 3- to 6-membered carbocycle.

14. The process of claim 13, wherein X is

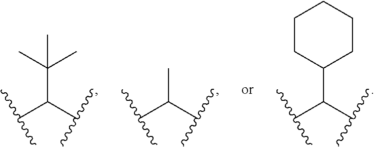

15. The process of claim 1, wherein R¹, R², R³, and R⁴ are each C₁₋₄alkyl.

16. The process of claim 15, wherein R¹, R², R³, and R⁴ are each —CH₃.

17. The process of claim 1, wherein R⁵ and R⁶ are each independently

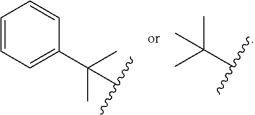

18. The process of claim 1, wherein R⁷ and R⁸ are each C₁₋₄alkyl or the unsubstituted or substituted phenyl.

19. The process of claim 18, wherein R⁷ and R⁸ are each —CH₃ or unsubstituted phenyl.

20. The process of claim 1, wherein the fluorophosphite ligand of formula (I) is a ligand of formula (Ia):

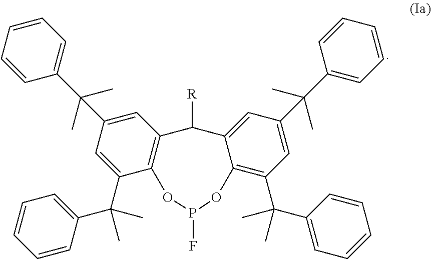

21. The process of claim 1, wherein the fluorophosphite ligand of formula (I) is a ligand of formula (Ib):

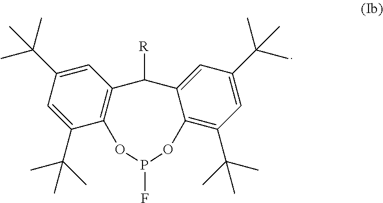

22. The process of claim 1, wherein the fluorophosphite ligand of formula (I) is [chemical structure]
